(19) United States
(12) Patent Application Publication
(10) Pub. No.: US 2026/0317546 A1
(43) Pub. Date: Oct. 15, 2026

(54) PRETARGETED BIMODAL IMAGING AGENTS

(71) Applicant: KØBENHAVNS UNIVERSITET, København K (DK)

(72) Inventors: Markus STAUDT, København K (DK); Matthias Manfred HERTH, København K (DK); Andreas KJÆR,, København K (DK)

(21) Appl. No.: 19/483,384

(22) PCT Filed: May 8, 2024
(86) PCT No.: PCT/EP2024/062817

§ 371 Date: Nov. 11, 2025

(30) Foreign Application Priority Data
May 11, 2023 (EP) 23172857.7

(51) Int. Cl. C09B 23/12; A61K 41/00; A61K 49/00; C09B 23/04; C09B 23/14; C09B 57/10

(52) U.S. Cl. CPC C09B 23/12; A61K 41/0057; A61K 49/0032; C09B 23/04; C09B 23/145; C09B 57/10

(57) ABSTRACT

The present invention relates to ¹⁸F-labelled compounds for use in imaging and/or diagnostics. In particular, the invention relates to water-soluble ¹⁸F-radiolabelled BODIPY tetrazines with suitable pharmacokinetics for in vivo pretargeted imaging and use thereof. Furthermore, the present invention relates to a method of imaging a biological structure comprising the ¹⁸F-radiolabelled BODIPY tetrazines.

### TECHNICAL FIELD OF THE INVENTION

The present invention relates to ¹⁸F-labelled compounds for use in imaging and/or diagnostics. In particular, the present invention relates to pretargeted immunoimaging of vectors, such as antibodies, with ¹⁸F-radiolabelled tetrazines.

### BACKGROUND OF THE INVENTION

Fluorescence intraoperative guided surgery as well as photodynamic therapies are gaining increasing attention within academic and clinical settings due to the inherent non-toxic properties of the employed fluorophores. Due to their nonspecific biodistribution, these fluorophores are often covalently linked to well-established receptor-targeting vectors like peptides, small molecules, antibody fragments or antibodies to reach the desired target sites. Given the changed pharmacokinetics exhibited after attaching the fluorophores, clinical translation is often hampered by insufficient biodistribution data being available. Proper target accumulation and therefore treatment success cannot be properly accessed before initiating the therapy.

A study by Carlson et al. has shown that connecting tetrazine to a bioorthogonal boron dipyrromethene (BODIPY) fluorophore enables exceptionally efficient energy transfer and quenching. Tetrazine-induced quenching of BODIPY fluorophore scaffolds were found to result in excellent turn-on rates upon reaction with TCO functionalities (1). Not only does it exhibit excellent turn-on properties upon reaction with TCO, the BODIPY moiety also allows for isotopic 18F-19F exchange (2). However, the methyl tetrazines have incompatible in vivo kinetics (3), and state of the art scaffolds are too apolar to be used in vivo.

WO 2022/189304 A2 and Edem Patricia E. et al. disclose radiolabelled tetrazines used in PET imaging, such as 68Ga-labelled tetrazines, however said compounds do not comprise a BODIPY structural unit.

Hence, an improved radiolabelled compound, which is in vivo compatible and allows for minimizing patient derived radiation doses, while harnessing the excellent target selectivities of vectors, such as monoclonal antibodies, would be advantageous.

### SUMMARY OF THE INVENTION

The invention presented relates to water-soluble ¹⁸F-radiolabelled BODIPY tetrazines with suitable pharmacokinetics for in vivo pretargeted imaging.

Thus, an object of the present invention relates to the provision of an improved ¹⁸F-radiolabelled BODIPY tetrazine, which allows for successful in vivo pretargeted fluorescence and imaging of biological structures, such as a tumor, using trans-cyclooctene (TCO)-labelled vectors.

In particular, it is an object of the present invention to provide a compound that solves the above mentioned problems of the prior art with a ¹⁸F-radiolabelled BODIPY tetrazine, which allows for minimizing patient derived radiation doses, while harnessing the excellent target selectivities of labelled vectors, such as antibodies.

Thus, a first aspect of the invention relates to a compound represented by the formula (1):

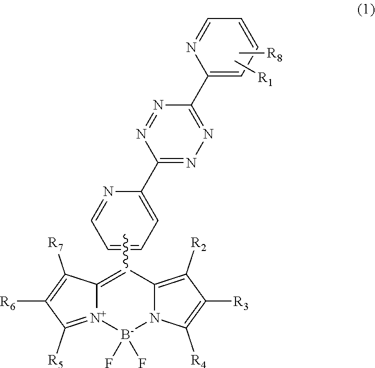

wherein R₁ is selected from a group consisting of a hydrogen, —CH₂N(CH₂COOH)₂, a hydroxy group, a sulfonamide, a sulfonic acid or its salt, a carboxyl group, a sulfonyl group, amine, a substituted amine with 1-5 polyethylene glycol unit(s), a —(O—CH₂—CH₂)₁₋₅—OCH₂—COOH, alkyl, preferably R₁ is in para position,
R₂ is selected from a group consisting of a hydrogen or alkyl,
R₃ is selected from a group consisting of a hydrogen, alkyl, a nitro group, a cyano group, a sulfonic acid or its salt, a carboylic acid, an amide, a sulfonamide, —CH₂N(CH₂COOH)₂ a halogen or a radioactive halogen, such as fluorine-18, iodine-123, iodine-124, iodine-125, iodine-131, astatine-211,
R₄ is selected from a group consisting of hydrogen, alkyl, —CH═CH(C₆H₄)R_{4′,}
wherein R_{4′} is selected from a group consisting of —N(R_{4′}R_{4′″})₂ or —OR_{4″}, wherein R_{4″} and R_{4′″} are independently selected from a group consisting of alkyl, —CH₂COOH, —(CH₂—CH₂—O)₁₋₅—CH₂—COOH, 1-5 polyethylene units, fluorine-18 or fluorine-19 labelled groups such as (CH₂)₁₋₆—F,
R₅ is selected from a group consisting of hydrogen, alkyl, —CH═CH(C₆H₄)R_{5′},
wherein R_{5′} is selected from a group consisting of —N(R_{5′}R_{5′″})₂ or —OR_{5″}, wherein R_{5′} and R_{5′″} are independently selected from a group consisting of alkyl, —CH₂COOH, —(CH₂—CH₂—O)₁₋₅—CH₂—COOH, 1-5 polyethylene units, fluorine-18 or fluorine-19 labelled groups such as (CH₂)₁₋₆—F,
R₆ is selected from a group consisting of a hydrogen, alkyl, a nitro group, a cyano group, a sulfonic acid or its salt, a carboylic acid, an amide, a halogen, a sulfonamide, —CH₂N(CH₂COOH)₂,
R₇ is selected from a group consisting of a hydrogen or alkyl, and
R₈ is selected from a group consisting of hydrogen, a fluorine-18 or fluorine-19 labelled groups such as —O—(CH₂)₁₋₆—F and —(CH₂)₁₋₆—F, a halogen or a radioactive halogen, such as fluorine-18, iodine-123, iodine-124, iodine-125, iodine-131, astatine-211;
wherein the wavy bond between the pyrridine and the [1,3,2]diazaborinine is in meta or para position on the pyrridine.

A second aspect of the invention relates to the compound according the first aspect for use in PET imaging, SPECT imaging and/or diagnostics.

A third aspect of the invention relates to the compound according the first aspect for use in intraoperative guided surgery.

A fourth aspect of the invention relates to the compound according the first aspect for use in photodynamic or photothermal therapy.

A fifth aspect of the invention relates to use of a compound according to the first aspect for PET imaging, SPECT imaging and/or diagnostics.

A sixth aspect of the present invention relates to a method of imaging a biological structure of a subject, the method comprising
administering a vector and the compound according to the first aspect,
imaging a contact between the vector and the compound in the biological structure.

### BRIEF DESCRIPTION OF THE FIGURES

FIG. 1 shows the in vivo click performance of the compounds of the present invention using anti-TAG72 mAb CC49 modified with TCO-tags (CC49-TCO) and [111In]In-DOTA-PEG11-BisPy-Tz. Increased in vivo performance is reflected by lower tumor uptake.

FIG. 2 shows the in vivo performance of a BODIPY-tetrazine of the present invention using ex vivo fluorescence measurements of selected organs.

FIG. 3 shows a descriptive picture of the pretargeted bimodal 18F-radiolabelled, turn-on BODIPY tetrazines and how they work in vivo.

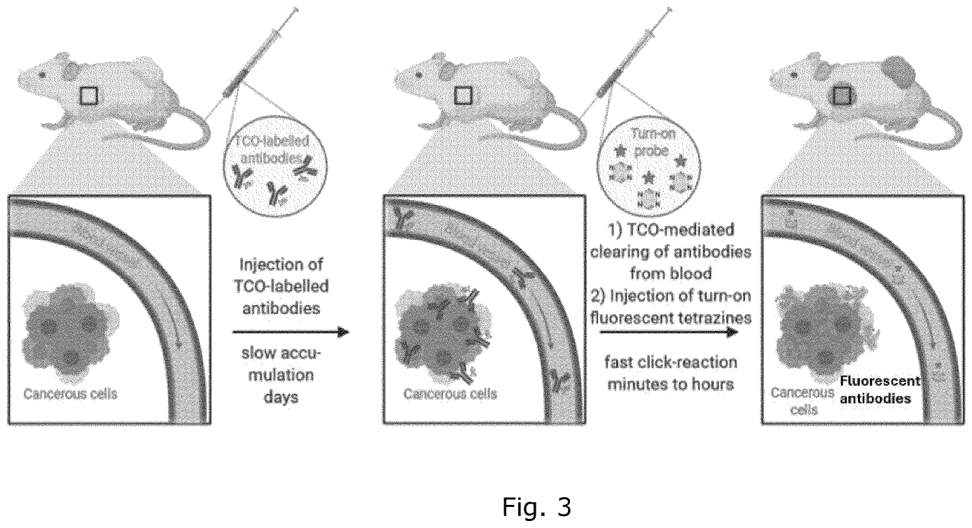

The present invention will now be described in more detail in the following.

### DETAILED DESCRIPTION OF THE INVENTION

### Definitions

Prior to discussing the present invention in further details, the following terms and conventions will first be defined:

### Vector

Vectors are peptides, small molecules, antibody fragments, single-chain variable fragments, antibodies, nanobodies, and affibodies capable of reaching the desired target sites. The vectors may be labelled, such as TCO-labelled.

### Intraoperative Guided Surgery.

A technique for visualizing biological structures, such as cancer tumors, during surgical procedures. Intraoperative guided surgery may be fluorescence intraoperative guided surgery, which has inherent non-toxic properties of the employed fluorophores.

### Photodynamic Therapy

Photodynamic therapy (PDT) is a treatment that involves light-sensitive medicine and a light source to destroy abnormal cells. A photosensitizer is a drug used in photodynamic therapy. When absorbed by cancer cells and exposed to light, the drug produces reactive oxygen species and kills the cancer cells.

### Photothermal Therapy

Photothermal therapy (PTT) is a technique that convert light into heat energy to kill the cancer cells. It is a therapeutic method with low toxicity, minimal invasiveness, and outstanding spatial-temporal selectivity. By irradiating photothermal agents under near-infrared (NIR) light, hyperthermia can be triggered to kill cancer cells in target tissues by energy transfer.

### AE105-TCO

AE105-TCO, a TCO-labelled nonapeptide with the sequence TCO-D-Cha-F-s-r-Y-L-W-S, has been successfully used in quantitative PET imaging. AE105 is a small linear peptide with high binding affinity to urokinase-type plasminogen activator receptor (uPAR), which is overexpressed in a variety of human cancers.

### BODIPY Tetrazine Compound

An aspect of the invention relates to a compound represented by the formula (1):

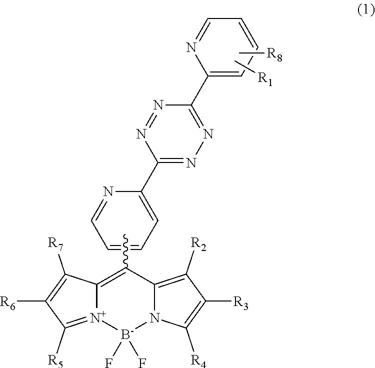

wherein R₁ is selected from a group consisting of a hydrogen, —CH₂N(CH₂COOH)₂, a hydroxy group, a sulfonamide, a sulfonic acid or its salt, a carboxyl group, a sulfonyl group, amine, a substituted amine with 1-5 polyethylene glycol unit(s), a
—(O—CH₂—CH₂)₁₋₅—OCH₂—COOH, alkyl, preferably R₁ is in para position,
R₂ is selected from a group consisting of a hydrogen or alkyl,
R₃ is selected from a group consisting of a hydrogen, alkyl, a nitro group, a cyano group, a sulfonic acid or its salt, a carboylic acid, an amide, a sulfonamide, —CH₂N(CH₂COOH)₂ a halogen or a radioactive halogen, such as fluorine-18, iodine-123, iodine-124, iodine-125, iodine-131, astatine-211,
R₄ is selected from a group consisting of hydrogen, alkyl, —CH═CH(C₆H₄)R_{4′},
wherein R_{4′} is selected from a group consisting of —N(R_{4″}R_{4′″})₂ or —OR_{4″}, wherein R_{4″} and R_{4″″} are independently selected from a group consisting of alkyl, —CH₂COOH, —(CH₂—CH₂—O)₁₋₅—CH₂—COOH, 1-5 polyethylene units, fluorine-18 or fluorine-19 labelled groups such as (CH₂)₁₋₆—F,
R₅ is selected from a group consisting of hydrogen, alkyl, —CH═CH(C₆H₄)R_{5′},
wherein R_{5′} is selected from a group consisting of —N(R_{5′}R_{5′″})₂ or —OR_{5″}, wherein R_{5′} and R_{5′″} are independently selected from a group consisting of alkyl, —CH₂COOH, —(CH₂—CH₂—O)₁₋₅—CH₂—COOH, 1-5 polyethylene units, fluorine-18 or fluorine-19 labelled groups such as (CH₂)₁₋₆—F,
R₆ is selected from a group consisting of a hydrogen, alkyl, a nitro group, a cyano group, a sulfonic acid or its salt, a carboylic acid, an amide, a halogen, a sulfonamide, —CH₂N(CH₂COOH)₂,
R₇ is selected from a group consisting of a hydrogen or alkyl, and
R₈ is selected from a group consisting of hydrogen, a fluorine-18 or fluorine-19 labelled groups such as —O—(CH₂)₁₋₆—F and —(CH₂)₁₋₆—F, a halogen or a radioactive halogen, such as fluorine-18, iodine-123, iodine-124, iodine-125, iodine-131, astatine-211;
wherein the wavy bond between the pyrridine and the [1,3,2]diazaborinine is in meta or para position on the pyrridine, as the bond in any of these two positions result in excellent fluorescence turn-on effect upon ligation with a TCO moiety.

In general in relation to the BODIPY tetrazines of the present invention, the R₁ group may be a polar group, as this contributes to the overall polarity and water-solubility of the compound. The polarity allows for successful in vivo pretargeted fluorescence and PET imaging.

The halogen on R₃ and R₆ is a photosensitizer used in photodynamic therapy. When absorbed by cancer cells and exposed to light, the photosensitizer agent produces singlet oxygen species and kills the cancer cells.

The substituents R₂-R₇ on the [1,3,2]diazaborinine group may shift the fluorescence of the group towards the red spectra depending on the nature and size of these substituents. Preferably, R₄ and R₅ on the [1,3,2]diazaborinine group may shift the fluorescence of the group towards the red spectra depending on the nature and size of these substituents.

As mentioned, the bond between the pyrridine and the [1,3,2]diazaborinine may be in meta position on the pyrridine, hence in an embodiment, the compound is further represented by the formula (2):

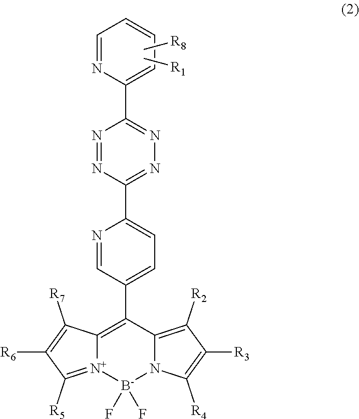

wherein R₁ is selected from a group consisting of a hydrogen, —CH₂N(CH₂COOH)₂, a hydroxy group, a sulfonamide, a sulfonic acid or its salt, a carboxyl group, a sulfonyl group, amine, a substituted amine with 1-5 polyethylene glycol unit(s), a —(O—CH₂—CH₂)₁₋₅—OCH₂—COOH, alkyl, preferably R₁ is in para position,
R₂ is selected from a group consisting of a hydrogen or alkyl,
R₃ is selected from a group consisting of a hydrogen, alkyl, a nitro group, a cyano group, a sulfonic acid or its salt, a carboylic acid, an amide, a halogen, a sulfonamide, —CH₂N(CH₂COOH)₂,
R₃ is selected from a group consisting of a hydrogen, alkyl, a nitro group, a cyano group, a sulfonic acid or its salt, a carboylic acid, an amide, a sulfonamide, —CH₂N(CH₂COOH)₂ a halogen or a radioactive halogen, such as fluorine-18, iodine-123, iodine-124, iodine-125, iodine-131, astatine-211,
R₄ is selected from a group consisting of hydrogen, alkyl, —CH═CH(C₆H₄)R_{4′},
wherein R_{4′} is selected from a group consisting of —N(R_{4″}R_{4′″})₂ or —OR_{4″}, wherein R_{4″} and R_{4″″} are independently selected from a group consisting of alkyl, —CH₂COOH, —(CH₂—CH₂—O)₁₋₅—CH₂—COOH, 1-5 polyethylene units, fluorine-18 or fluorine-19 labelled groups such as (CH₂)₁₋₆—F,
R₅ is selected from a group consisting of hydrogen, alkyl, —CH═CH(C₆H₄)R_{5′},
wherein R_{5′} is selected from a group consisting of —N(R_{5′} R_{5′″})₂ or —OR_{5″}, wherein R_{5″} and R_{5′″} are independently selected from a group consisting of alkyl, —CH₂COOH, —(CH₂—CH₂—O)₁₋₅—CH₂—COOH, 1-5 polyethylene units, fluorine-18 or fluorine-19 labelled groups such as (CH₂)₁₋₆—F,
R₆ is selected from a group consisting of a hydrogen, alkyl, a nitro group, a cyano group, a sulfonic acid or its salt, a carboylic acid, an amide, a halogen, a sulfonamide, —CH₂N(CH₂COOH)₂,
R₇ is selected from a group consisting of a hydrogen or alkyl, and
R₈ is selected from a group consisting of hydrogen, a fluorine-18 or fluorine-19 labelled groups such as —O—(CH₂)₁₋₆—F and —(CH₂)₁₋₆—F, a halogen or a radioactive halogen, such as fluorine-18, iodine-123, iodine-124, iodine-125, iodine-131, astatine-211.

As mentioned, R₅ may be —CH═CH(C₆H₄)R_{5′}, hence in an embodiment, the compound is further represented by the formula (3):

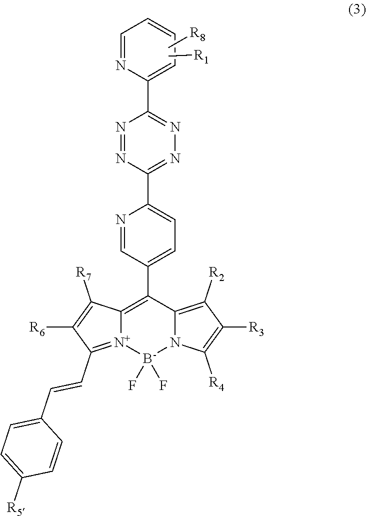

wherein R₁ is selected from a group consisting of a hydrogen, —CH₂N(CH₂COOH)₂, a hydroxy group, a sulfonamide, a sulfonic acid or its salt, a carboxyl group, a sulfonyl group, amine, a substituted amine with 1-5 polyethylene glycol unit(s), a —(O—CH₂—CH₂)₁₋₅—OCH₂—COOH, alkyl, preferably R₁ is in para position,
R₂ is selected from a group consisting of a hydrogen or alkyl,
R₃ is selected from a group consisting of a hydrogen, alkyl, a nitro group, a cyano group, a sulfonic acid or its salt, a carboylic acid, an amide, a sulfonamide, —CH₂N(CH₂COOH)₂ a halogen or a radioactive halogen, such as fluorine-18, iodine-123, iodine-124, iodine-125, iodine-131, astatine-211,
R₄ is selected from a group consisting of hydrogen, alkyl, —CH═CH(C₆H₄)R_{4′,}
wherein R_{4′} is selected from a group consisting of —N(R_{4″}R_{4′″})₂ or —OR_{4″}, wherein R_{4″} and R_{4′″} are independently selected from a group consisting of alkyl, —CH₂COOH, —(CH₂—CH₂—O)₁₋₅—CH₂—COOH, 1-5 polyethylene units, fluorine-18 or fluorine-19 labelled groups such as (CH₂)₁₋₆—F,
R_{5′} is selected from a group consisting of —N(R_{5″}R_{5′″})₂ or —OR_{5″}, wherein R_{5′} and R_{5′″} are independently selected from a group consisting of alkyl, —CH₂COOH, —(CH₂—CH₂—O)₁₋₅—CH₂—COOH, 1-5 polyethylene unit(s), fluorine-18 or fluorine-19 labelled groups such as (CH₂)₁₋₆—F,
R₆ is selected from a group consisting of a hydrogen, alkyl, a nitro group, a cyano group, a sulfonic acid or its salt, a carboylic acid, an amide, a halogen, a sulfonamide, —CH₂N(CH₂COOH)₂,
R₇ is selected from a group consisting of a hydrogen or alkyl, and
R₈ is selected from a group consisting of hydrogen, a fluorine-18 or fluorine-19 labelled groups such as —O—(CH₂)₁₋₆—F and —(CH₂)₁₋₆—F, a halogen or a radioactive halogen, such as fluorine-18, iodine-123, iodine-124, iodine-125, iodine-131, astatine-211.

As mentioned, R₄ may be —CH═CH(C₆H₄)R_{4′} and R₅ may be —CH═CH(C₆H₄)R_{5′}, hence in an embodiment, thethe compound is further represented by the formula (4):

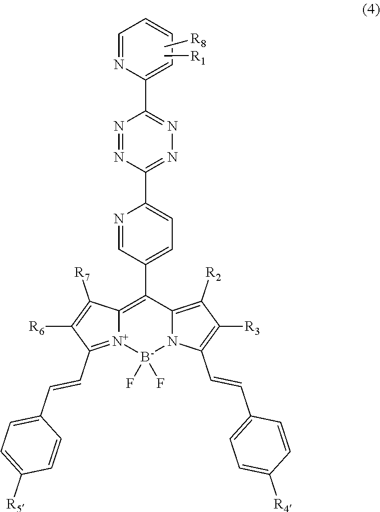

wherein R₁ is selected from a group consisting of a hydrogen, —CH₂N(CH₂COOH)₂, a hydroxy group, a sulfonamide, a sulfonic acid or its salt, a carboxyl group, a sulfonyl group, amine, a substituted amine with 1-5 polyethylene glycol unit(s), a —(O—CH₂—CH₂)₁₋₅—OCH₂—COOH, alkyl, preferably R₁ is in para position,
R₂ is selected from a group consisting of a hydrogen or alkyl,
R₃ is selected from a group consisting of a hydrogen, alkyl, a nitro group, a cyano group, a sulfonic acid or its salt, a carboylic acid, an amide, a sulfonamide, —CH₂N(CH₂COOH)₂ a halogen or a radioactive halogen, such as fluorine-18, iodine-123, iodine-124, iodine-125, iodine-131, astatine-211,
R_{4′} is selected from a group consisting of —N(R_{4″}R_{4′″})₂ or —OR_{4″}, wherein R_{4″} and R_{4″″} are independently selected from a group consisting of alkyl, —CH₂COOH, —(CH₂—CH₂—O)₁₋₅—CH₂—COOH, 1-5 polyethylene units, fluorine-18 or fluorine-19 labelled groups such as (CH₂)₁₋₆—F,
R_{5′} is selected from a group consisting of —N(R_{5″}R_{5′″})₂ or —OR_{5″}, wherein R_{5′} and R_{5′″} are independently selected from a group consisting of alkyl, —CH₂COOH, —(CH₂—CH₂—O)₁₋₅—CH₂—COOH, 1-5 polyethylene unit(s), fluorine-18 or fluorine-19 labelled groups such as (CH₂)₁₋₆—F,
R₆ is selected from a group consisting of a hydrogen, alkyl, a nitro group, a cyano group, a sulfonic acid or its salt, a carboylic acid, an amide, a halogen, a sulfonamide, —CH₂N(CH₂COOH)₂,
R₇ is selected from a group consisting of a hydrogen or alkyl, and
R₈ is selected from a group consisting of hydrogen, a fluorine-18 or fluorine-19 labelled groups such as —O—(CH₂)₁₋₆—F and —(CH₂)₁₋₆—F, a halogen or a radioactive halogen, such as fluorine-18, iodine-123, iodine-124, iodine-125, iodine-131, astatine-211.

As mentioned, R₁ may be —CH₂N(CH₂COOH)₂, hence in an embodiment, the compound is further represented by the formula (5):

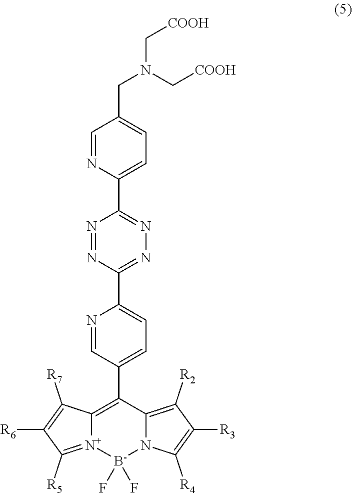

R₂ is selected from a group consisting of a hydrogen or alkyl,
R₃ is selected from a group consisting of a hydrogen, alkyl, a nitro group, a cyano group, a sulfonic acid or its salt, a carboylic acid, an amide, a sulfonamide, —CH₂N(CH₂COOH)₂ a halogen or a radioactive halogen, such as fluorine-18, iodine-123, iodine-124, iodine-125, iodine-131, astatine-211,
R₄ is selected from a group consisting of hydrogen, alkyl, —CH═CH(C₆H₄)R_{4′, wherein R}_{4′} is selected from a group consisting of —N(R_{4″}R_{4′″})₂ or —OR_{4″}, wherein R_{4″} and R_{4′″} are independently selected from a group consisting of alkyl, —CH₂COOH, —(CH₂—CH₂—O)₁₋₅—CH₂—COOH, 1-5 polyethylene units, fluorine-18 or fluorine-19 labelled groups such as (CH₂)₁₋₆—F,
R₅ is selected from a group consisting of hydrogen, alkyl, —CH═CH(C₆H₄)R_{5′}, wherein R_{5′} is selected from a group consisting of —N(R_{5″}R_{5′″})₂ or —OR_{5″}, wherein R_{5′} and R_{5′″} are independently selected from a group consisting of alkyl, —CH₂COOH, —(CH₂—CH₂—O)₁₋₅—CH₂—COOH, 1-5 polyethylene units, fluorine-18 or fluorine-19 labelled groups such as (CH₂)₁₋₆—F,
R₆ is selected from a group consisting of a hydrogen, alkyl, a nitro group, a cyano group, a sulfonic acid or its salt, a carboylic acid, an amide, a halogen, a sulfonamide, —CH₂N(CH₂COOH)₂, and
R₇ is selected from a group consisting of a hydrogen or alkyl.

As mentioned, R₁ may be —CH₂N(CH₂COOH)₂ and R₅ may be —CH═CH(C₆H₄)R_{5′}, hence in an embodiment, the compound is further represented by the formula (6):

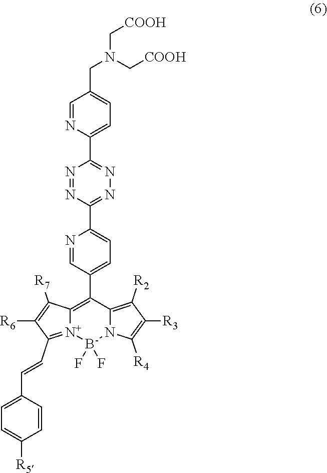

R₂ is selected from a group consisting of a hydrogen or alkyl,
R₃ is selected from a group consisting of a hydrogen, alkyl, a nitro group, a cyano group, a sulfonic acid or its salt, a carboylic acid, an amide, a sulfonamide, —CH₂N(CH₂COOH)₂ a halogen or a radioactive halogen, such as fluorine-18, iodine-123, iodine-124, iodine-125, iodine-131, astatine-211,
R₄ is selected from a group consisting of hydrogen, alkyl, —CH═CH(C₆H₄)R_{4′, wherein R}_{4′} is selected from a group consisting of —N(R_{4″}R_{4′″})₂ or —OR_{4″}, wherein
R_{4″} and R_{4′″} are independently selected from a group consisting of alkyl, —CH₂COOH, —(CH₂—CH₂—O)₁₋₅—CH₂—COOH, 1-5 polyethylene units, fluorine-18 or fluorine-19 labelled groups such as (CH₂)₁₋₆—F,
R_{5′} is selected from a group consisting of —N(R_{5″}R_{5′″})₂ or —OR_{5″}, wherein R_{5′} and R_{5′″} are independently selected from a group consisting of alkyl, —CH₂COOH, —(CH₂—CH₂—O)₁₋₅—CH₂—COOH, 1-5 polyethylene unit(s), fluorine-18 or fluorine-19 labelled groups such as (CH₂)₁₋₆—F,
R₆ is selected from a group consisting of a hydrogen, alkyl, a nitro group, a cyano group, a sulfonic acid or its salt, a carboylic acid, an amide, a halogen, a sulfonamide, —CH₂N(CH₂COOH)₂, and
R₇ is selected from a group consisting of a hydrogen or alkyl.

As mentioned, R₁ may be —CH₂N(CH₂COOH)₂, R₄ may be —CH═CH(C₆H₄)R_{4′, and R}₅ may be —CH═CH(C₆H₄)R_{5′}, hence in an embodiment, the compound is further represented by the formula (7):

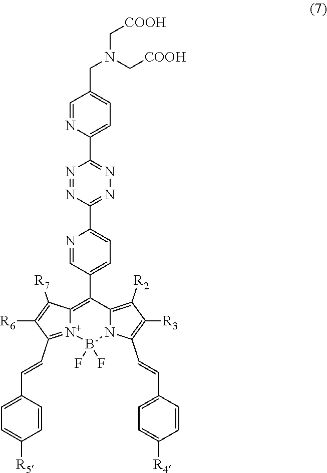

R₂ is selected from a group consisting of a hydrogen or alkyl,
R₃ is selected from a group consisting of a hydrogen, alkyl, a nitro group, a cyano group, a sulfonic acid or its salt, a carboylic acid, an amide, a sulfonamide, —CH₂N(CH₂COOH)₂ a halogen or a radioactive halogen, such as fluorine-18, iodine-123, iodine-124, iodine-125, iodine-131, astatine-211,
R_{4′} is selected from a group consisting of —N(R_{4″}R_{4′″})₂ or —OR_{4″}, wherein R_{4″} and R_{4′″} are independently selected from a group consisting of alkyl, —CH₂COOH, —(CH₂—CH₂—O)₁₋₅—CH₂—COOH, 1-5 polyethylene units, fluorine-18 or fluorine-19 labelled groups such as (CH₂)₁₋₆—F,
R_{5′} is selected from a group consisting of —N(R_{5″}R_{5′″})₂ or —OR_{5″}, wherein R_{5′} and R_{5′″} are independently selected from a group consisting of alkyl, —CH₂COOH, —(CH₂—CH₂—O)₁₋₅—CH₂—COOH, 1-5 polyethylene unit(s), fluorine-18 or fluorine-19 labelled groups such as (CH₂)₁₋₆—F,
R₆ is selected from a group consisting of a hydrogen, alkyl, a nitro group, a cyano group, a sulfonic acid or its salt, a carboylic acid, an amide, a halogen, a sulfonamide, —CH₂N(CH₂COOH)₂, and
R₇ is selected from a group consisting of a hydrogen or alkyl.

As mentioned, the bond between the pyrridine and the [1,3,2]diazaborinine may be in para position on the pyrridine, hence in an embodiment, the compound is further represented by the formula (8):

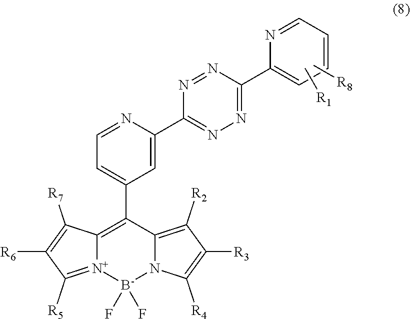

wherein R₁ is selected from a group consisting of a hydrogen, —CH₂N(CH₂COOH)₂, a hydroxy group, a sulfonamide, a sulfonic acid or its salt, a carboxyl group, a sulfonyl group, amine, a substituted amine with 1-5 polyethylene glycol unit(s), a —(O—CH₂—CH₂)₁₋₅—OCH₂—COOH, alkyl, preferably R₁ is in para position,
R₂ is selected from a group consisting of a hydrogen or alkyl,
R₃ is selected from a group consisting of a hydrogen, alkyl, a nitro group, a cyano group, a sulfonic acid or its salt, a carboylic acid, an amide, a sulfonamide, —CH₂N(CH₂COOH)₂ a halogen or a radioactive halogen, such as fluorine-18, iodine-123, iodine-124, iodine-125, iodine-131, astatine-211,
R₄ is selected from a group consisting of hydrogen, alkyl, —CH═CH(C₆H₄)R_{4′,}
wherein R_{4′} is selected from a group consisting of —N(R_{4″}R_{4′″})₂ or —OR_{4″}, wherein R_{4″} and R_{4′″} are independently selected from a group consisting of alkyl, —CH₂COOH, —(CH₂—CH₂—O)₁₋₅—CH₂—COOH, 1-5 polyethylene units, fluorine-18 or fluorine-19 labelled groups such as (CH₂)₁₋₆—F,
R₅ is selected from a group consisting of hydrogen, alkyl, —CH═CH(C₆H₄)R_{5′}, wherein R_{5′} is selected from a group consisting of —N(R_{5″}R_{5′″})₂ or —OR_{5″}, wherein R_{5′} and R_{5′″} are independently selected from a group consisting of alkyl, —CH₂COOH, —(CH₂—CH₂—O)₁₋₅—CH₂—COOH, 1-5 polyethylene units, fluorine-18 or fluorine-19 labelled groups such as (CH₂)₁₋₆—F,
R₆ is selected from a group consisting of a hydrogen, alkyl, a nitro group, a cyano group, a sulfonic acid or its salt, a carboylic acid, an amide, a halogen, a sulfonamide, —CH₂N(CH₂COOH)₂,
R₇ is selected from a group consisting of a hydrogen or alkyl, and
R₈ is selected from a group consisting of hydrogen, a fluorine-18 or fluorine-19 labelled groups such as —O—(CH₂)₁₋₆—F and —(CH₂)₁₋₆—F, a halogen or a radioactive halogen, such as fluorine-18, iodine-123, iodine-124, iodine-125, iodine-131, astatine-211.

As mentioned, R₅ may be —CH═CH(C₆H₄)R_{5′}, hence in an embodiment, the compound is further represented by the formula (9):

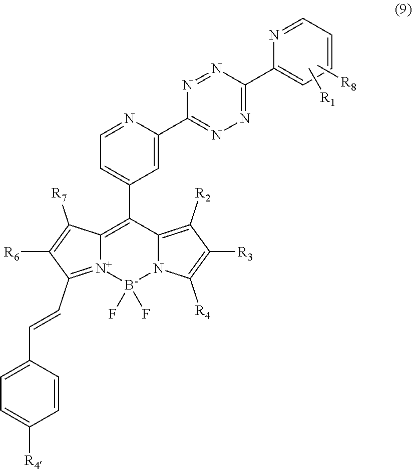

wherein R₁ is selected from a group consisting of a hydrogen, —CH₂N(CH₂COOH)₂, a hydroxy group, a sulfonamide, a sulfonic acid or its salt, a carboxyl group, a sulfonyl group, amine, a substituted amine with 1-5 polyethylene glycol unit(s), a —(O—CH₂—CH₂)₁₋₅—OCH₂—COOH, alkyl, preferably R₁ is in para position,
R₂ is selected from a group consisting of a hydrogen or alkyl,
R₃ is selected from a group consisting of a hydrogen, alkyl, a nitro group, a cyano group, a sulfonic acid or its salt, a carboylic acid, an amide, a sulfonamide, —CH₂N(CH₂COOH)₂ a halogen or a radioactive halogen, such as fluorine-18, iodine-123, iodine-124, iodine-125, iodine-131, astatine-211,
R₄ is selected from a group consisting of hydrogen, alkyl, —CH═CH(C₆H₄)R_{4′, wherein R}_{4′} is selected from a group consisting of —N(R_{4″}R_{4′″})₂ or —OR_{4″}, wherein
R_{4″} and R_{4″″} are independently selected from a group consisting of alkyl, —CH₂COOH, —(CH₂—CH₂—O)₁₋₅—CH₂—COOH, 1-5 polyethylene units, fluorine-18 or fluorine-19 labelled groups such as (CH₂)₁₋₆—F,
R_{5′} is selected from a group consisting of —N(R_{5′} R_{5′″})₂ or —OR_{5″}, wherein R_{5′} and R_{5′″} are independently selected from a group consisting of alkyl, —CH₂COOH, —(CH₂—CH₂—O)₁₋₅—CH₂—COOH, 1-5 polyethylene unit(s), fluorine-18 or fluorine-19 labelled groups such as (CH₂)₁₋₆—F,
R₆ is selected from a group consisting of a hydrogen, alkyl, a nitro group, a cyano group, a sulfonic acid or its salt, a carboylic acid, an amide, a halogen, a sulfonamide, —CH₂N(CH₂COOH)₂,
R₇ is selected from a group consisting of a hydrogen or alkyl, and
R₈ is selected from a group consisting of hydrogen, a fluorine-18 or fluorine-19 labelled groups such as —O—(CH₂)₁₋₆—F and —(CH₂)₁₋₆—F, a halogen or a radioactive halogen, such as fluorine-18, iodine-123, iodine-124, iodine-125, iodine-131, astatine-211.

As mentioned, R₄ may be —CH═CH(C₆H₄)R₄ and R₅ may be —CH═CH(C₆H₄)R_{5′}, hence in an embodiment, the compound is further represented by the formula (10):

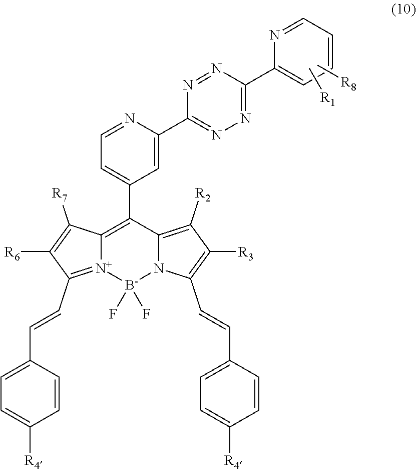

wherein R₁ is selected from a group consisting of a hydrogen, —CH₂N(CH₂COOH)₂, a hydroxy group, a sulfonamide, a sulfonic acid or its salt, a carboxyl group, a sulfonyl group, amine, a substituted amine with 1-5 polyethylene glycol unit(s), a —(O—CH₂—CH₂)₁₋₅—OCH₂—COOH, alkyl, preferably R₁ is in para position,
R₂ is selected from a group consisting of a hydrogen or alkyl,
R₃ is selected from a group consisting of a hydrogen, alkyl, a nitro group, a cyano group, a sulfonic acid or its salt, a carboylic acid, an amide, a sulfonamide, —CH₂N(CH₂COOH)₂ a halogen or a radioactive halogen, such as fluorine-18, iodine-123, iodine-124, iodine-125, iodine-131, astatine-211,
R_{4′} is selected from a group consisting of —N(R_{4″}R_{4′″})₂ or —OR_{4″}, wherein R_{4″} and R_{4″″} are independently selected from a group consisting of alkyl, —CH₂COOH, —(CH₂—CH₂—O)₁₋₅—CH₂—COOH, 1-5 polyethylene units, fluorine-18 or fluorine-19 labelled groups such as (CH₂)₁₋₆—F,
R_{5′} is selected from a group consisting of —N(R_{5′} R_{5′″})₂ or —OR_{5″}, wherein R_{5′} and R_{5′″} are independently selected from a group consisting of alkyl, —CH₂COOH, —(CH₂—CH₂—O)₁₋₅—CH₂—COOH, 1-5 polyethylene unit(s), fluorine-18 or fluorine-19 labelled groups such as (CH₂)₁₋₆—F,
R₆ is selected from a group consisting of a hydrogen, alkyl, a nitro group, a cyano group, a sulfonic acid or its salt, a carboylic acid, an amide, a halogen, a sulfonamide, —CH₂N(CH₂COOH)₂,
R₇ is selected from a group consisting of a hydrogen or alkyl, and
R₈ is selected from a group consisting of hydrogen, a fluorine-18 or fluorine-19 labelled groups such as —O—(CH₂)₁₋₆—F and —(CH₂)₁₋₆—F, a halogen or a radioactive halogen, such as fluorine-18, iodine-123, iodine-124, iodine-125, iodine-131, astatine-211.

R₁ may be —CH₂N(CH₂COOH)₂, hence in an embodiment, the compound is further represented by the formula (11):

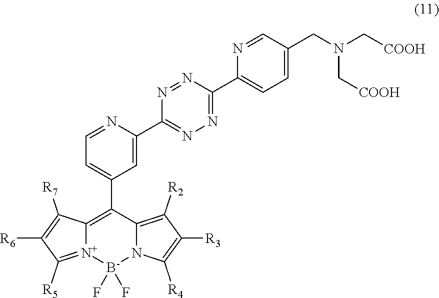

R₂ is selected from a group consisting of a hydrogen or alkyl,
R₃ is selected from a group consisting of a hydrogen, alkyl, a nitro group, a cyano group, a sulfonic acid or its salt, a carboylic acid, an amide, a sulfonamide, —CH₂N(CH₂COOH)₂ a halogen or a radioactive halogen, such as fluorine-18, iodine-123, iodine-124, iodine-125, iodine-131, astatine-211,
R₄ is selected from a group consisting of hydrogen, alkyl, —CH═CH(C₆H₄)R_{4′, wherein R}_{4′} is selected from a group consisting of —N(R_{4″}R_{4′″})₂ or —OR_{4″}, wherein R_{4″} and R_{4′″} are independently selected from a group consisting of alkyl, —CH₂COOH, —(CH₂—CH₂—O)₁₋₅—CH₂—COOH, 1-5 polyethylene units, fluorine-18 or fluorine-19 labelled groups such as (CH₂)₁₋₆—F,
R₅ is selected from a group consisting of hydrogen, alkyl, —CH═CH(C₆H₄)R_{5′}, wherein R_{5′} is selected from a group consisting of —N(R_{5″}R_{5′″})₂ or —OR_{5″}, wherein R_{5′} and R_{5′″} are independently selected from a group consisting of alkyl, —CH₂COOH, —(CH₂—CH₂—O)₁₋₅—CH₂—COOH, 1-5 polyethylene units, fluorine-18 or fluorine-19 labelled groups such as (CH₂)₁₋₆—F,
R₆ is selected from a group consisting of a hydrogen, alkyl, a nitro group, a cyano group, a sulfonic acid or its salt, a carboylic acid, an amide, a halogen, a sulfonamide, —CH₂N(CH₂COOH)₂, and
R₇ is selected from a group consisting of a hydrogen or alkyl.

R₁ may be —CH₂N(CH₂COOH)₂ and R₅ may be —CH═CH(C₆H₄)R_{5′}, hence in an embodiment, the compound is further represented by the formula (12):

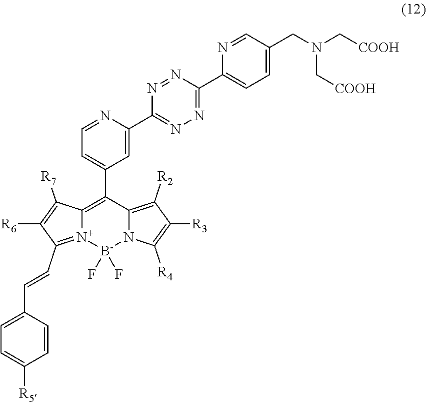

R₂ is selected from a group consisting of a hydrogen or alkyl,
R₃ is selected from a group consisting of a hydrogen, alkyl, a nitro group, a cyano group, a sulfonic acid or its salt, a carboylic acid, an amide, a sulfonamide, —CH₂N(CH₂COOH)₂ a halogen or a radioactive halogen, such as fluorine-18, iodine-123, iodine-124, iodine-125, iodine-131, astatine-211,
R₄ is selected from a group consisting of hydrogen, alkyl, —CH═CH(C₆H₄)R_{4′, wherein R}_{4′} is selected from a group consisting of —N(R_{4″}R_{4′″})₂ or —OR_{4″}, wherein R_{4″} and R_{4′″} are independently selected from a group consisting of alkyl, —CH₂COOH, —(CH₂—CH₂—O)₁₋₅—CH₂—COOH, 1-5 polyethylene units, fluorine-18 or fluorine-19 labelled groups such as (CH₂)₁₋₆—F,
R_{5′} is selected from a group consisting of —N(R_{5″}R_{5′″})₂ or —OR_{5″}, wherein R_{5′} and R_{5′″} are independently selected from a group consisting of alkyl, —CH₂COOH, —(CH₂—CH₂—O)₁₋₅—CH₂—COOH, 1-5 polyethylene unit(s), fluorine-18 or fluorine-19 labelled groups such as (CH₂)₁₋₆—F,
R₆ is selected from a group consisting of a hydrogen, alkyl, a nitro group, a cyano group, a sulfonic acid or its salt, a carboylic acid, an amide, a halogen, a sulfonamide, —CH₂N(CH₂COOH)₂, and
R₇ is selected from a group consisting of a hydrogen or alkyl.

As mentioned, R₁ may be —CH₂N(CH₂COOH)₂, R₄ may be —CH═CH(C₆H₄)R_{4′, and R}₅ may be —CH═CH(C₆H₄)R_{5′}, hence in an embodiment, the compound is further represented by the formula (13):

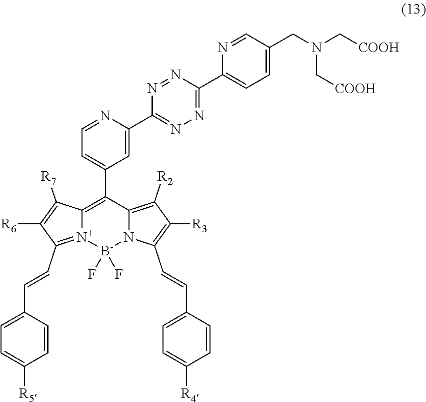

R₂ is selected from a group consisting of a hydrogen or alkyl,
R₃ is selected from a group consisting of a hydrogen, alkyl, a nitro group, a cyano group, a sulfonic acid or its salt, a carboylic acid, an amide, a sulfonamide, —CH₂N(CH₂COOH)₂ a halogen or a radioactive halogen, such as fluorine-18, iodine-123, iodine-124, iodine-125, iodine-131, astatine-211,
R_{4′} is selected from a group consisting of —N(R_{4″}R_{4′″})₂ or —OR_{4″}, wherein R_{4″} and R_{4′″} are independently selected from a group consisting of alkyl, —CH₂COOH, —(CH₂—CH₂—O)₁₋₅—CH₂—COOH, 1-5 polyethylene units, fluorine-18 or fluorine-19 labelled groups such as (CH₂)₁₋₆—F,
R_{5′} is selected from a group consisting of —N(R_{5″}R_{5′″})₂ or —OR_{5″}, wherein R_{5′} and R_{5′″} are independently selected from a group consisting of alkyl, —CH₂COOH, —(CH₂—CH₂—O)₁₋₅—CH₂—COOH, 1-5 polyethylene unit(s), fluorine-18 or fluorine-19 labelled groups such as (CH₂)₁₋₆—F,
R₆ is selected from a group consisting of a hydrogen, alkyl, a nitro group, a cyano group, a sulfonic acid or its salt, a carboylic acid, an amide, a halogen, a sulfonamide, —CH₂N(CH₂COOH)₂, and
R₇ is selected from a group consisting of a hydrogen or alkyl.

In an embodiment, the compound is selected from a group consisting of:

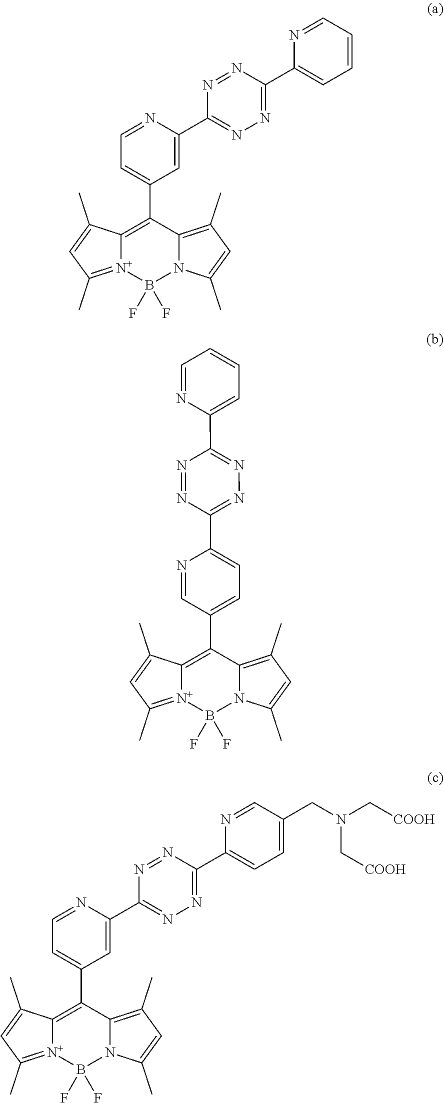

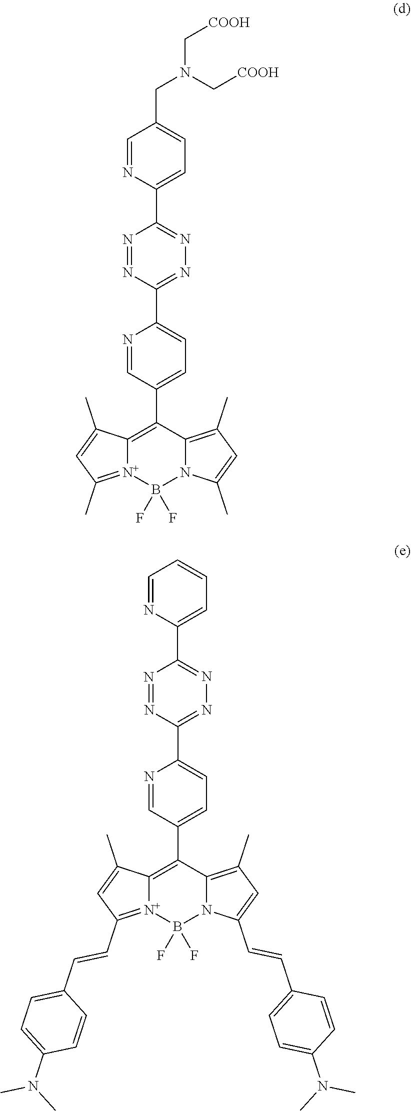

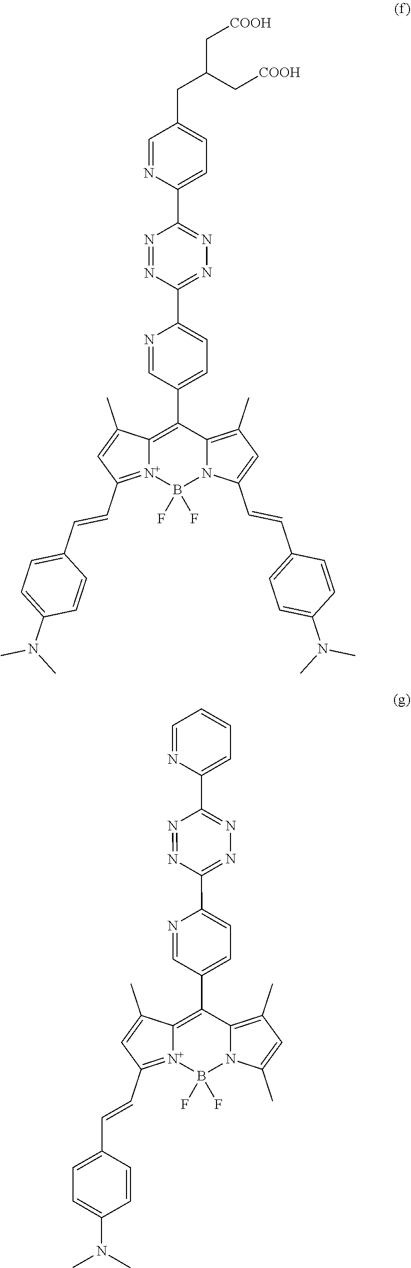

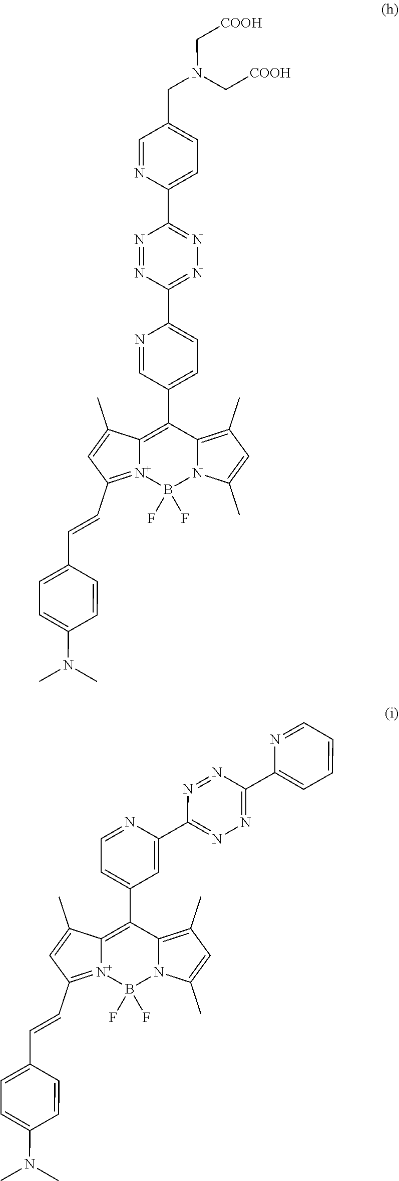

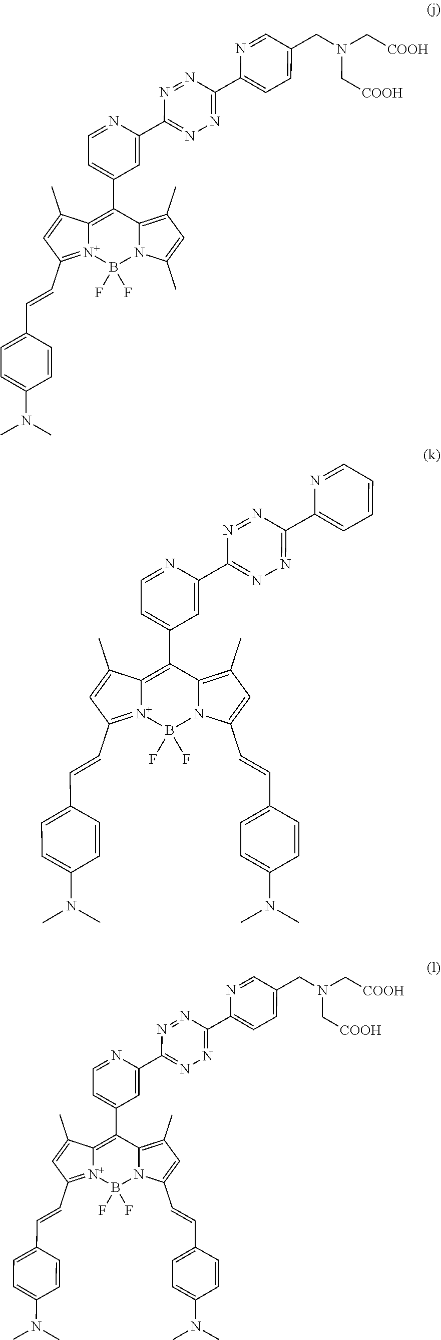

The synthesis of these compounds are shown in Example 1.

In a preferred embodiment, the compound has a blocking effect of at least 90% as seen in Example 2.

### Use of the BODIPY Tetrazine Compound

A second aspect of the invention relates to the compound according the first aspect for use in PET imaging, SPECT imaging and/or diagnostics.

A third aspect of the invention relates to the compound according the first aspect for use in intraoperative guided surgery.

A fourth aspect of the invention relates to the compound according the first aspect for use in photodynamic or photothermal therapy.

A fifth aspect of the invention relates to use of a compound according to the first aspect for PET imaging, SPECT imaging and/or diagnostics.

### A Method of Imaging a Biological Structure

A sixth aspect of the invention relates to a method of imaging a biological structure of a subject, the method comprising
administering a vector and the compound according to the first aspect; and
imaging a contact between the vector and the compound in the biological structure.

In an embodiment, the vector is a labelled vector, in order to ensure specificity between the vector and the tetrazine compound of the first aspect.

In an embodiment, the labelled vector is a trans-cyclooctene (TCO)-labelled antibody, nanobodies, affibodies, single-chain variable fragments, peptides or small molecules, such as CC49-TCO, trastuzumab-TCO, PSMA-TCO or AE105-TCO (TCO-D-Cha-F-s-r-Y-L-W-S). The TCO reacts with the tetrazine compound of the first aspect, which is based on an inverse electron-demand Diels-Alder cycloaddition reaction between the TCO and tetrazine reaction pair, forming a dihydropyridazine. The combination of ultrafast kinetics, selectivity, and bioorthogonality makes TCO-Tz the ideal pair in low concentration applications. In an embodiment, the contact between the vector and the compound is a click reaction.

In a preferred embodiment, the biological structure is a tumor.

In a preferred embodiment, the subject is mammal, preferably human.

In an embodiment, the imaging is PET or SPECT imaging.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

### Example 1—Synthesis of BODIPY Tetrazines

### Compound IUPAC Names

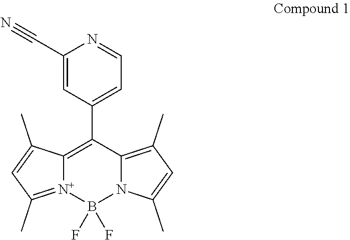

4-(5,5-difluoro-1,3,7,9-tetramethyl-5H-4λ4,5λ4-dipyrrolo[1,2-c:2′,1′-f][1,3,2]diazaborinin-10-yl)picolinonitrile

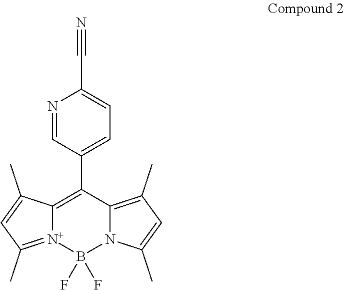

5-(5,5-difluoro-1,3,7,9-tetramethyl-5H-4λ4,5λ4-dipyrrolo[1,2-c:2′,1′-f][1,3,2]diazaborinin-10-yl)picolinonitrile

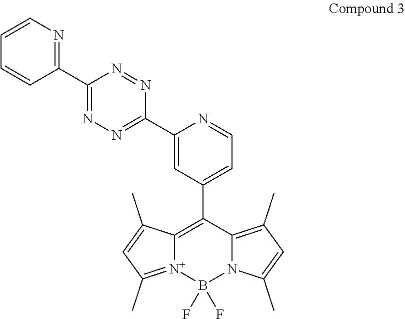

5,5-difluoro-1,3,7,9-tetramethyl-10-(2-(6-(pyridin-2-yl)-1,2,4,5-tetrazin-3-yl)pyridin-4-yl)-5H-4λ4,5λ4-dipyrrolo[1,2-c:2′,1′-f][1,3,2]diazaborinine

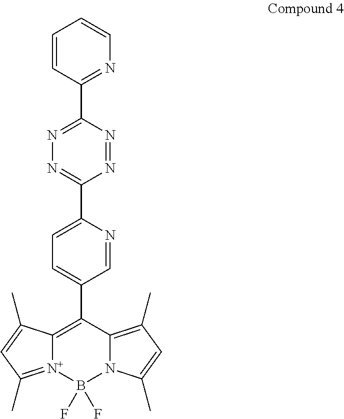

5,5-difluoro-1,3,7,9-tetramethyl-10-(6-(6-(pyridin-2-yl)-1,2,4,5-tetrazin-3-yl)pyridin-3-yl)-5H-4λ4,5λ4-dipyrrolo[1,2-c:2′,1′-f][1,3,2]diazaborinine

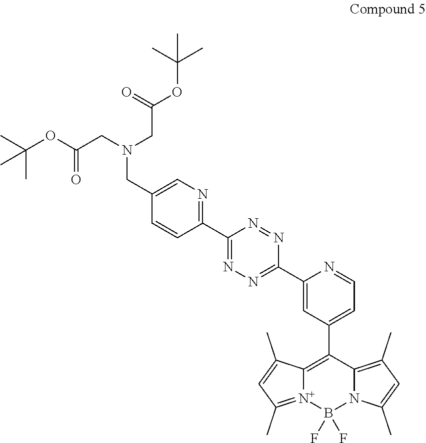

di-tert-butyl 2,2′-(((6-(6-(4-(5,5-difluoro-1,3,7,9-tetramethyl-5H-4λ4,5λ4-dipyrrolo[1,2-c:2′,1′-f][1,3,2]diazaborinin-10-yl)pyridin-2-yl)-1,2,4,5-tetrazin-3-yl)pyridin-3-yl)methyl)azanediyl)diacetate Compound 6:

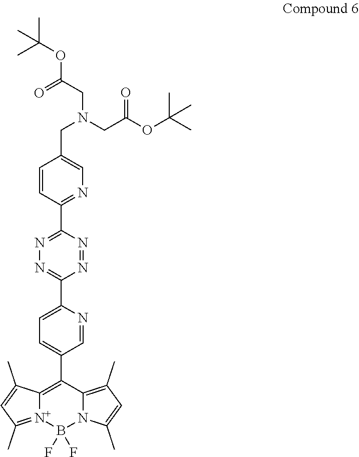

di-tert-butyl 2,2′-(((6-(6-(5-(5,5-difluoro-1,3,7,9-tetramethyl-5H-4λ4,5λ4-dipyrrolo[1,2-c:2′,1′-f][1,3,2]diazaborinin-10-yl)pyridin-2-yl)-1,2,4,5-tetrazin-3-yl)pyridin-3-yl)methyl)azanediyl)diacetate

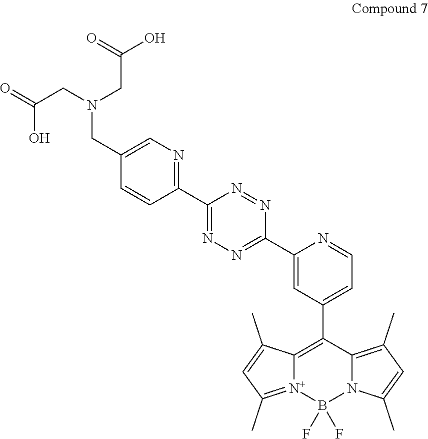

2,2′-(((6-(6-(4-(5,5-difluoro-1,3,7,9-tetramethyl-5H-4λ4,5AN4-dipyrrolo[1,2-c:2′,1′-f][1,3,2]diazaborinin-10-yl)pyridin-2-yl)-1,2,4,5-tetrazin-3-yl)pyridin-3-yl)methyl)azanediyl)diacetic acid

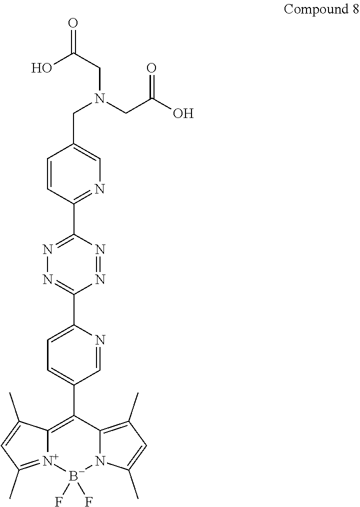 [SMILES: CC1=CC(C)=[N+]2C1=C(c1ccc(-c3nnc(-c4ccc(CN(CC(=O)O)CC(=O)O)cn4)nn3)nc1)c1c(C)cc(C)n1[B-]2(F)F]

2,2′-(((6-(6-(5-(5,5-difluoro-1,3,7,9-tetramethyl-5H-4λ4,5λ4-dipyrrolo[1,2-c:2′,1′-f][1,3,2]diazaborinin-10-yl)pyridin-2-yl)-1,2,4,5-tetrazin-3-yl)pyridin-3-yl)methyl)azanediyl)diacetic acid

### Materials and Methods

All reactions requiring inert conditions were performed under argon atmosphere in flamedried glassware. Commercially available chemicals were used without further purification.DCM, THE and DMF were dried using a SG WATER solvent purification system from PureProcess Technology. Remaining anhydrous solvents were prepared by storing them over molecular sieve for at least 48 h. Rf values and reaction controls were performed on F254silica gel 60 thin-layer chromatography alumina sheets from Merck. Chromatographic purification was performed using either flash chromatography on silica gel 60 Å (40-63 μm) or Combiflash. NMR-spectra were acquired using a 400 MHz Bruker Avance II equipped with a 5 mm broad band probe. Samples were dissolved in deuterated solvent and analysed at 300 K. Proton spectra, at 400.09 MHz, were acquired using 30°-pulses, a spectral width of 8 kHz, collecting 16 scans with a length of 65536 data points with a relaxation delay of 1.0 sec. FID's were zero-filled to twice the size and exponentially multiplied with a line broadening factor of 0.3 Hz before Fourier transform. Carbon spectra were acquired at 100.60 MHz with 30°-pulses, a spectral width of 24 kHz, collecting 256 scans with a length of 65536data points and with a relaxation delay of 2.0 sec. The 13C nuclei were 1H-decoupled using the Waltz-16 composite pulse decoupling scheme. FID's were exponentially multiplied with a line broadening factor of 1.0 Hz before Fourier transform. NMR-spectra were acquired using a 600 MHz Bruker Avance III HD equipped with a cryogenically cooled 5 mm dual probe optimized for 13 C and 1H. Samples were dissolved in deuterated solvent and analysed at 300 K. Proton spectra, at 600.03 MHz, were acquired using 30°-pulses, a spectral width of 12 kHz, collecting 16 scans with a length of 65536 data points with a relaxation delay of 1.0 sec. FID's were zero-filled to twice the size and exponentially multiplied with a line broadening factor of 0.3 Hz before Fourier transform. Carbon spectra were acquired at 150.88 MHz with 30°-pulses, a spectral width of 36 kHz, collecting 256 scans with a length of 65536 data points and with a relaxation delay of 2.0 sec. The 13 C nuclei were 1H-decoupled using the Waltz-16 composite pulse decoupling scheme. FID's were exponentially multiplied with a line broadening factor of 1.0 Hz before Fourier transform.

### Preparation of 5,5-difluoro-1,3,7,9-tetramethyl-10-(2-(6-(pyridin-2-yl)-1,2,4,5-tetrazin-3-yl)pyridin-4-yl)-5H-4λ4,5λ4-dipyrrolo[1,2-c:2′,1′-f][1,3,2]diazaborinine (compound 3)

### Synthesis Overview

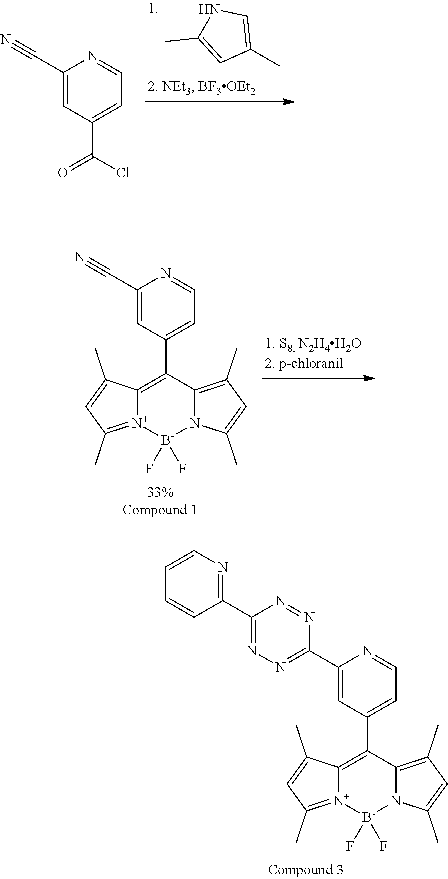

### Preparation of Compound 1

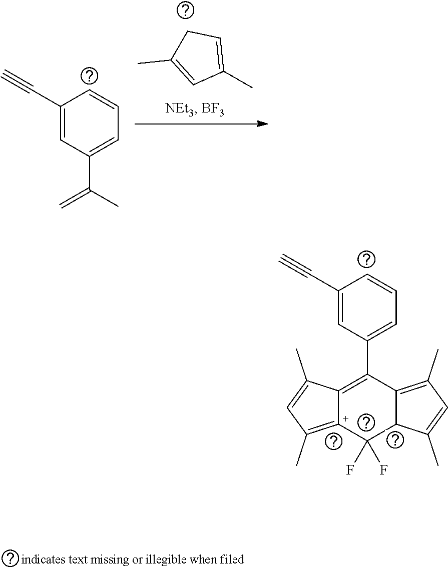

2-Cyanoisonicotinoyl chloride (442.2 mg, 2.67 mmol) was dissolved in 40 mL of absolute DCM under an argon atmosphere and 2,4-dimethylpyrrole (0.5 ml, 462.0 mg, 4.86 mmol) was added. The reaction mixture was stirred at rt overnight during which it turned dark red, followed by addition of triethylamine (2.5 mL) and dropwise addition of BF3·OEt2 (2.5 mL) at 0° C. The reaction was stirred over night and 50 mL of water were added to quench the reaction. The phases were seperated and the aqueous phase was extracted twice with 20 mL of DCM. The combined organic phases were washed with saturated aqueous Na2CO3 solution (3×20 mL), water (2×20 mL) and brine (20 mL) and the organic phases was dried over MgSO4. Removal of the solvent furnished a red-brown oil, which was purified by column chromatography (silica gel, toluene) furnishing a red solid (277.2 mg, 0.79 mmol, 33%).

1H NMR (400 MHz, CDCl3) δ 8.89 (d, J=4.9 Hz, 1H), 7.73 (t, J=1.6 Hz, 1H), 7.56 (dd, J=4.9, 1.6 Hz, 1H), 6.04 (s, 2H), 2.57 (s, 6H), 1.39 (s, 6H).

### Preparation of Compound 3

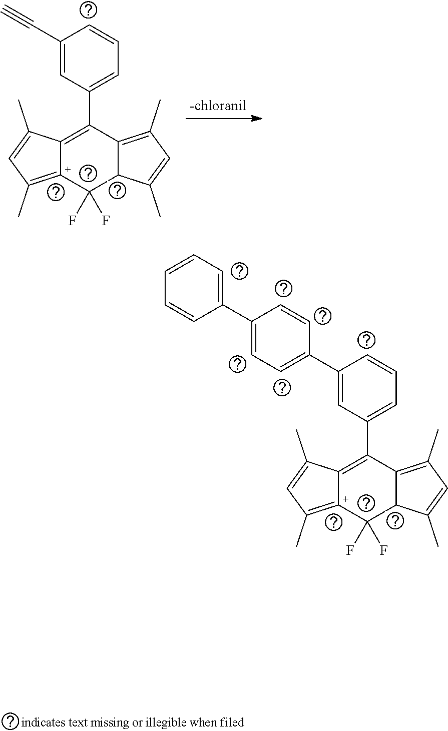

Compound 1 (250.0 mg, 0.71 mmol, 1.0 eq.), 2-cyanopyridine (1.04 g, 9.97 mmol, 14.0 eq.) and sulfur (91.3 mg, 0.36 mmol, 0.5 eq.) were suspended in 20.0 mL of EtOH under an argon atmosphere and hydrazine hydrate (1.04 mL, 1.07 g, 21.36 mmol, 30.0 eq.) were added dropwise. The reaction was heated to reflux, leading to rapid gas formation, which was allowed to escape by piercing the septum on top of the reflux condenser with two needles.

After 4 hours reflux, the reaction was cooled to room temperature and 100 mL of DCM and 100 mL of water were added. The phases were separated and the organic phase washed twice with 100 mL of water and once with 100 mL of brine. The organic phase was dried over MgSO4 and the solvent removed under reduced pressure furnishing a deep red solid.

The thus obtained crude product was purified by flash column chromatography (silica gel, 0 to 5% EtOAc in DCM v/v) yielding an orange solid.

The solid was redissolved in 5 mL of DCM and p-chloranil (28.4 mg, 0.12 mmol) was added. The reaction mixture was stirred for 10 minutes and celite was added. The solvent was removed under reduced pressure and the crude products purified by automated flash column chromatography (silica gel, 0 to 50% EtOAc in DCM, v/v). The product was obtained as a deep orange solid (25.0 mg, 5.2 μmol, 7%). 1H NMR (400 MHz, CDCl3) δ 9.15 (d, J=4.9 Hz, 1H), 9.03-8.97 (m, 1H), 8.81-8.74 (m, 2H), 8.03 (td, J=7.8, 1.7 Hz, 1H), 7.64-7.57 (m, 2H), 6.04 (s, 2H), 2.58 (s, 6H), 1.48 (s, 6H).

### Preparation of 2,2′-(((6-(6-(4-(5,5-difluoro-1,3,7,9-tetramethyl-5H-4λ4,5λ4-dipyrrolo[1,2-c:2′,1′-f][1,3,2]diazaborinin-10-yl)pyridin-2-yl)-1,2,4,5-tetrazin-3-yl)pyridin-3-yl)methyl)azanediyl)diacetic acid (compound 7)

### Synthesis Overview

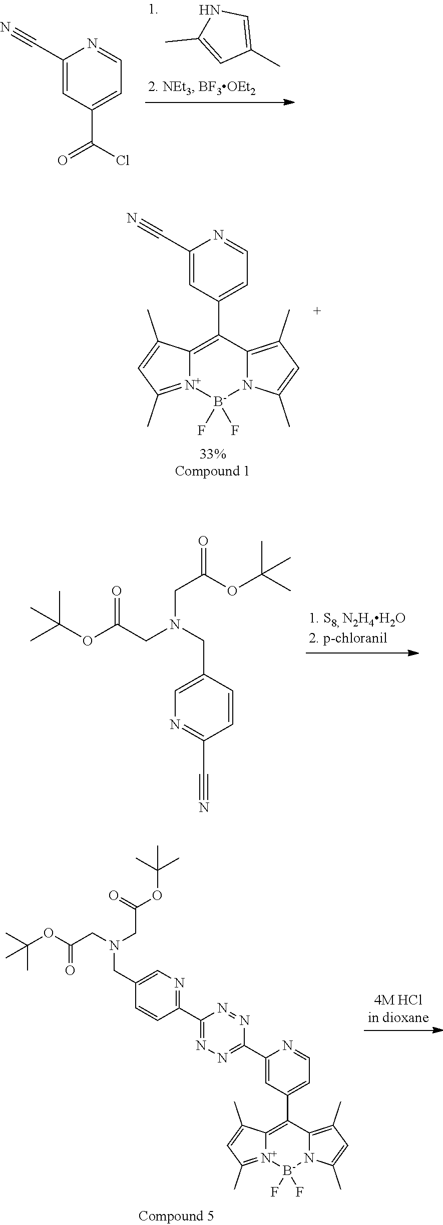

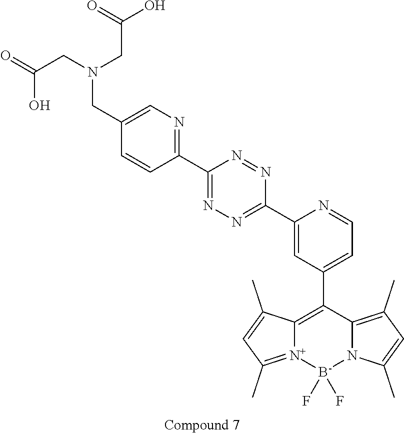 [SMILES: CC1=CC(C)=[N+]2C1=C(c1ccnc(-c3nnc(-c4ccc(CN(CC(=O)O)CC(=O)O)cn4)nn3)c1)c1c(C)cc(C)n1[B-]2(F)F]

### Preparation of Compound 1

As described above.

### Preparation of Compound 5

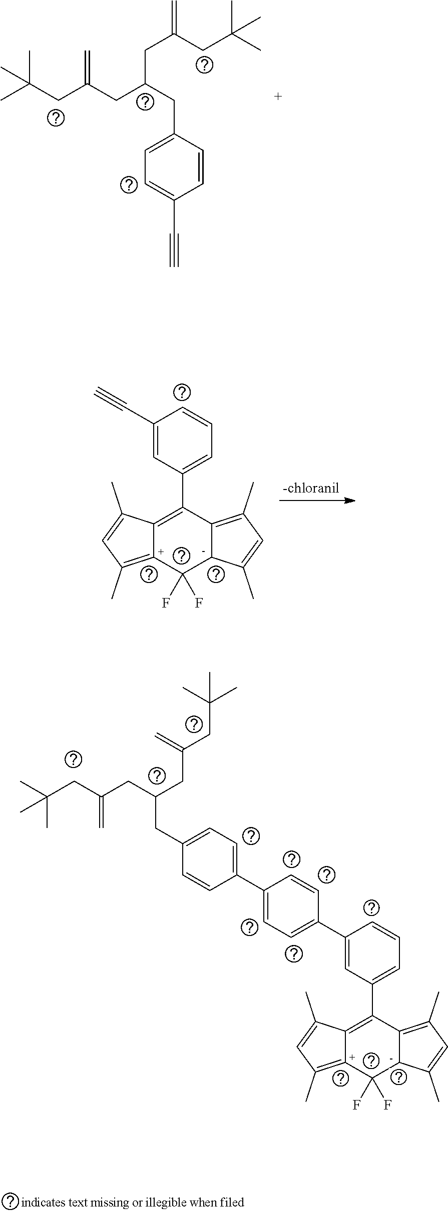

Compound 1 (100.0 mg, 0.28 mmol, 1.0 eq.), di-tert-butyl 2,2′-(((6-cyanopyridin-3-yl)methyl)azanediyl)diacetate (1.03 g, 2.85 mmol, 10.0 eq.) and sulfur (36.5 mg, 0.14 mmol, 0.5 eq.) were suspended in 10.0 mL of pOH under an argon atmosphere and hydrazine hydrate (416.4 μL, 427.6 mg, 8.54 mmol, 30.0 eq.) were added dropwise. The reaction was heated to reflux, leading to rapid gas formation, which was allowed to escape by piercing the septum on top of the reflux condenser with two needles.

After 4 hours reflux, the reaction was cooled to room temperature and 40 mL of DCM and 40 mL of water were added. After adding a small amount of brine to aid in phase separation, the phases were separated and the organic phase washed twice with 40 mL of water and once with 40 mL of brine. The organic phase was dried over MgSO4 and the solvent removed under reduced pressure furnishing a deep red solid.

To remove the majority of polar side products, the crude product was redissolved in 20 mL of DCM and poured into heptane. The formed solids were filtered off and the filtrate concentrated under vacuum.

The thus obtained crude product was purified by flash column chromatography (silica gel, 0 to 4% EtOAc in DCM v/v) yielding an orange oil.

The obtained oil (228.4 mg) was dissolved in 5 mL of DCM and p-chloranil (83.2 mg) was added. The solution was stirred for 10 minutes at rt. The solvent was removed under reduced pressure and the crude product purified by automated flash column chromatography (silica gel, EtOAc in DCM, 0 to 30% v/v). Red solid (46.3 mg, 62.6 μmol, 22%).

1H NMR (400 MHz, CDCl3) δ 9.13 (d, J=4.9 Hz, 1H), 8.90 (d, J=2.0 Hz, 1H), 8.76 (s, 1H), 8.72 (d, J=8.1 Hz, 1H), 8.21 (dd, J=8.1, 2.1 Hz, 1H), 7.59 (dd, J=4.8, 1.6 Hz, 1H), 6.03 (s, 2H), 4.09 (s, 2H), 3.47 (s, 4H), 2.57 (s, 6H), 1.49-1.43 (m, 24H).

19F NMR (376 MHz, CDCl3) δ-146.08 (q, J=32.6 Hz), −146.17 (q, J=32.6 Hz). 11B NMR (128 MHz, CDCl3) δ 0.71 (t, J=32.6 Hz).

### Preparation of Compound 7

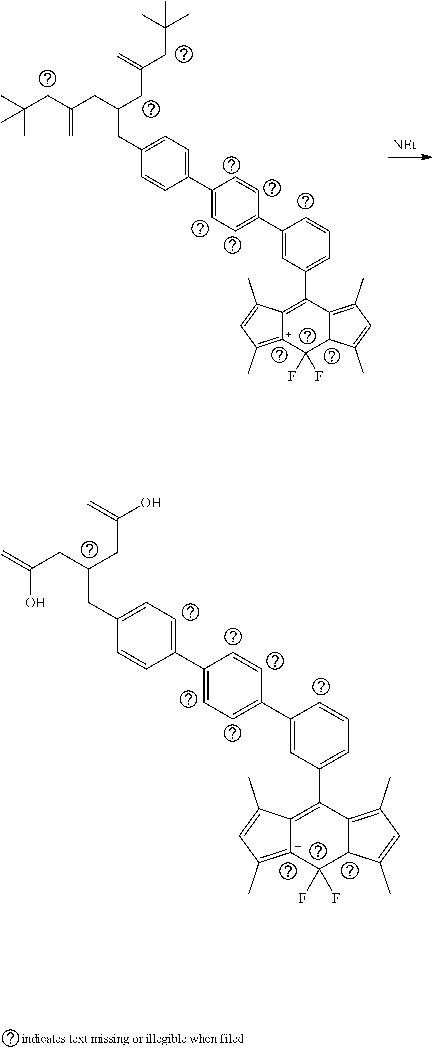

Compound 5 (11.4 mg, 15.4 μmol) was dissolved in 2 mL of 4 M HCl in dioxane at 0° C. The reaction mixture was warmed to room temperature and stirred for 4 hours. The solvent was removed under reduced pressure furnishing a red solid. The crude product was redissolved in 5 mL of MeCN:H2O (1:1 v/v, 0.1% TFA) and purified by preparative HPLC furnishing a red solid (1.9 mg, 3.0 μmol, 20%). 1H NMR (600 MHz, MeOD) δ 9.09 (dd, J=4.9, 0.9 Hz, 1H), 8.95 (d, J=2.5 Hz, 1H), 8.82-8.76 (m, 2H), 8.28 (dd, J=8.1, 2.2 Hz, 1H), 7.85 (dd, J=4.9, 1.6 Hz, 1H), 6.15 (s, 2H), 4.24 (s, 2H), 3.71 (s, 4H), 2.52 (s, 6H), 1.53 (s, 6H). 11B NMR (128 MHz, MeOD) b 4.65 (t, J=31.9 Hz).

13C NMR (151 MHz, MeOD) b 173.6, 164.9, 164.8, 158.1, 152.7, 152.6, 152.4, 150.4, 146.9, 144.1, 140.6, 138.8, 138.5, 131.5, 128.2, 125.6, 125.6, 123.1, 56.4, 55.4, 15.2, 14.6.

19F NMR (376 MHz, MeOD) δ-143.09 (q, J=31.5 Hz).

### Preparation of 5,5-difluoro-1,3,7,9-tetramethyl-10-(6-(6-(pyridin-2-yl)-1,2,4,5-tetrazin-3-yl)pyridin-3-yl)-5H-4λ4,5λ4-dipyrrolo[1,2-c:2′,1′-f][1,3,2]diazaborinine (compound 4)

### Synthesis Overview

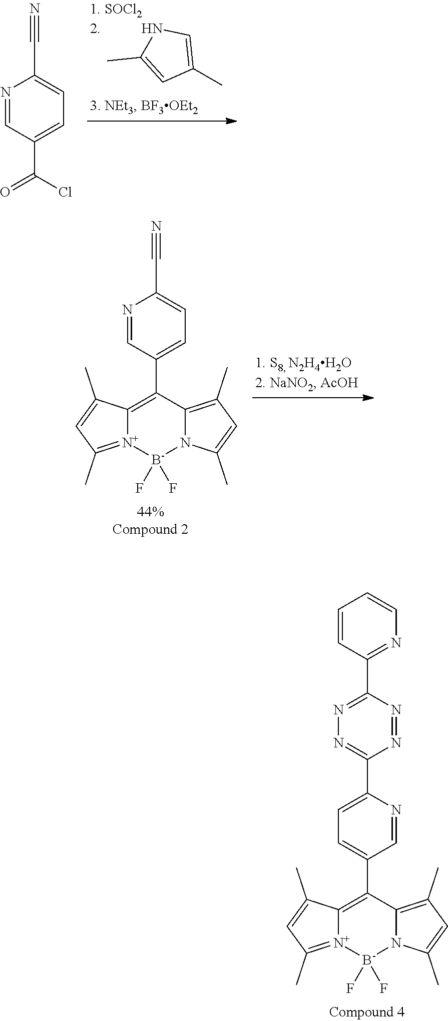

### Preparation of Compound 2

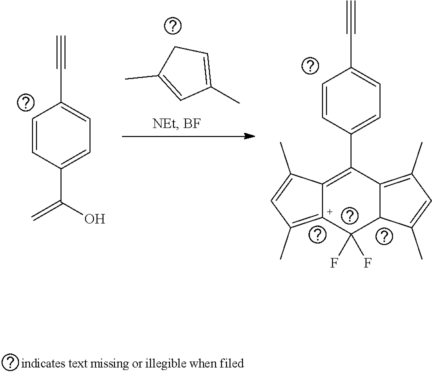

6-cyanonicotinic acid (1.2 g, 8.10 mmol, 1.0 equiv.) was suspended in 10 mL of anhydrous toluene and thionyl chloride (2.29 g, 1.5 mL, 19.04 mmol, 2.35 equiv.) was added. The reaction mixture was heated under reflux for 18 hours and the solvents were removed under reduced pressure. The crude product was redissolved in 150 mL of DCM and 2,4-dimethylpyrrole (1.54 g, 1.67 mL, 16.20 mmol, 2.0 equiv.) was added. The resulting solution was stirred over night during which it turned deep red. The reaction mixture was cooled to 0° C. in an ice bath and trimethylamine (6.05 g, 8.34 mL, 59.8 mmol, 7.38 equiv.) were added, followed by dropwise addition of BF3·OEt2 (9.43 g, 8.34 mL, 66.41 mmol, 8.20 equiv.). After stirring over night, the deep orange reaction mixture was quenched by addition of 100 mL of 2N NaCO3 solution. After stirring for 10 minutes, the phases were separated and the organic phases washed with 2N NaCO3 solution (2×100 mL), water (3×100 mL) and brine (100 mL). The organic phases was dried over MgSO4 and the solvent removed under reduced pressure. Flash column chromatography (silica gel, toluene) furnished a red solid (1.25 g, 3.56 mmol, 44%).

1H NMR (400 MHz, CDCl3) δ 8.79-8.63 (m, 1H), 7.94-7.77 (m, 2H), 6.04 (s, 2H), 2.56 (s, 6H), 1.36 (s, 6H).

13C NMR (101 MHz, CDCl3) δ 157.5, 150.4, 142.4, 137.5, 135.1, 134.8, 134.5, 131.1, 128.5, 122.4, 116.8, 15.4, 14.8.

### Preparation of Compound 4

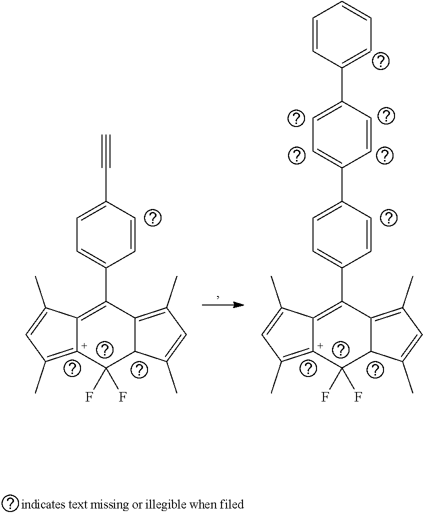

Compound 2 (200.0 mg, 0.67 mmol, 1.0 eq.), 2-cyanopyridine (118.6 mg, 1.14 mmol, 2.0 eq.) and sulfur (36.5 mg, 0.14 mmol, 0.25 eq.) were suspended in 1.2 mL of EtOH under an argon atmosphere and hydrazine hydrate (555.2 μL, 570.2 mg, 11.4 mmol, 20.0 eq.) were added dropwise. The reaction was heated to 90° C. After 3 hours the solution was cooled to rt and used in the oxidation step as is.

The reaction mixture was poured into 300 mg NaNO2 in 10 mL of water, the reaction vessel washed with DCM and poured into the aqueous mixture. AcOH (approx. 4 mL) were added carefully under heavy stirring. After 10 min 10 mL of DCM were added and the phases separated. The aqueous phase was extracted with 10 mL of DCM and the combined organic phases washed thrice with 15 mL of water. The organic phase was dried over MgSO4 and the solvent removed under reduced pressure furnishing a deep red solid. The crude product was purified using flash column chromatography on silica gel using an eluent of DCM to DCM:EtOAc 4:1 v/v. The desired product was obtained as a deep red solid (11.1 mg, 23.0 μmol, 8%).

1H NMR (400 MHz, CDCl3) δ 9.15 (d, J=4.9 Hz, 1H), 9.05-8.96 (m, 1H), 8.83-8.73 (m, 2H), 8.03 (td, J=7.8, 1.7 Hz, 1H), 7.69-7.53 (m, 2H), 6.04 (s, 2H), 2.58 (s, 6H), 1.48 (s, 6H).

11B NMR (128 MHz, CDCl3) δ 0.74 (t, J=32.3 Hz).

13C NMR (101 MHz, CDCl3) δ 164.1, 163.6, 157.1, 151.3, 150.7, 150.2, 150.0, 142.7, 138.0, 137.7, 136.1, 134.7, 131.4, 126.9, 124.9, 124.5, 122.2, 15.5, 14.8.

19F NMR (376 MHz, CDCl3) δ-146.07 (q, J=32.3 Hz), −146.16 (q, J=32.3 Hz).

### Preparation of 2,2′-(((6-(6-(5-(5,5-difluoro-1,3,7,9-tetramethyl-5H-4λ4,5λ4-dipyrrolo[1,2-c:2′,1′-f][1,3,2]diazaborinin-10-yl)pyridin-2-yl)-1,2,4,5-tetrazin-3-yl)pyridin-3-yl)methyl)azanediyl)diacetic acid (compound 8)

### Synthesis Overview

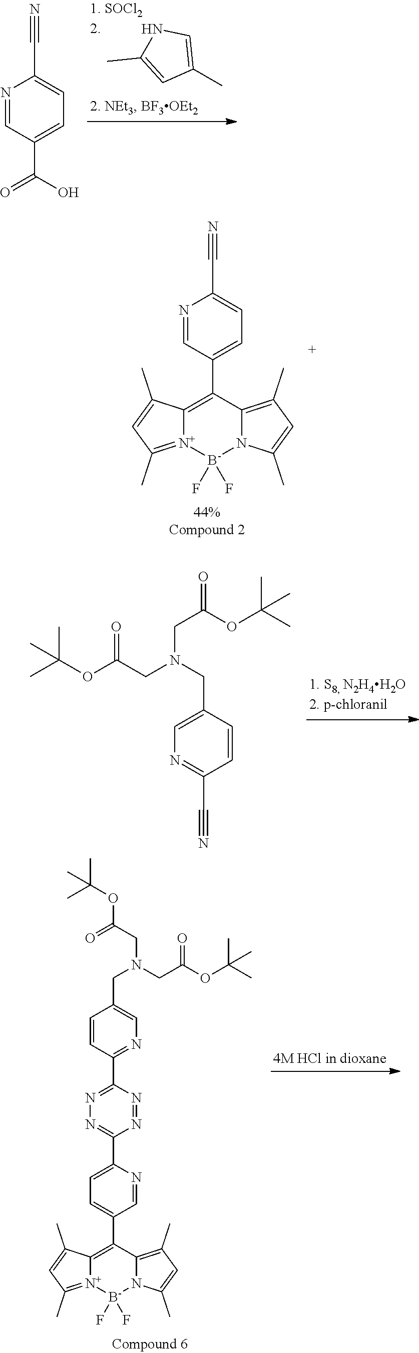

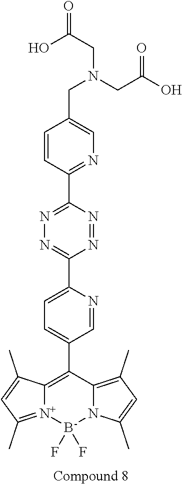 [SMILES: CC1=CC(C)=[N+]2C1=C(c1ccc(-c3nnc(-c4ccc(CN(CC(=O)O)CC(=O)O)cn4)nn3)nc1)c1c(C)cc(C)n1[B-]2(F)F]

### Preparation of Compound 2

As described above.

### Preparation of Compound 6

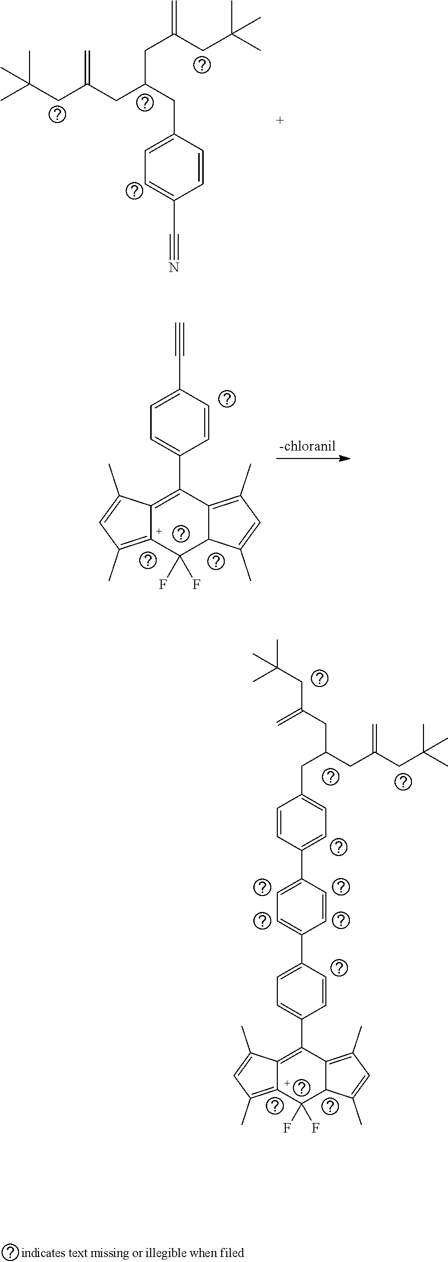

Compound 2 (100.0 mg, 0.28 mmol, 1.0 eq.), di-tert-butyl 2,2′-(((6-cyanopyridin-3-yl)methyl)azanediyl)diacetate (1.03 g, 2.85 mmol, 10.0 eq.) and sulfur (36.5 mg, 0.14 mmol, 0.5 eq.) were suspended in 10.0 mL of EtOH under an argon atmosphere and hydrazine hydrate (416.4 μL, 427.6 mg, 8.54 mmol, 30.0 eq.) were added dropwise. The reaction was heated to reflux, leading to rapid gas formation, which was allowed to escape by piercing the septum on top of the reflux condenser with two needles. After 4 hours reflux, the reaction was cooled to room temperature and 40 mL of DCM and 40 mL of water were added. After adding a small amount of brine to aid in phase separation, the phases were separated and the organic phase washed twice with 40 mL of water and once with 40 mL of brine. The organic phase was dried over MgSO4 and the solvent removed under reduced pressure furnishing a deep red solid.

The thus obtained crude product was purified by flash column chromatography (silica gel, 0 to 4% EtOAc in DCM v/v) yielding an orange oil.

The obtained oil (102.0 mg) was dissolved in 5 mL of DCM and p-chloranil (104.8 mg) was added. The solution was stirred for 10 minutes at rt. The solvent was removed under reduced pressure and the crude product purified by automated flash column chromatography (silica gel, EtOAc in DCM, 0 to 30% v/v). Red solid (65.0 mg, 87.8 μmol, 31%).

1H NMR (600 MHz, CDCl3) δ 8.96-8.90 (m, 3H), 8.74 (d, J=8.0 Hz, 1H), 8.24 (dd, J=8.1, 2.1 Hz, 1H), 8.00 (dd, J=8.0, 2.2 Hz, 1H), 6.04 (s, 2H), 4.12 (s, 2H), 3.49 (s, 4H), 2.58 (s, 6H), 1.49-1.46 (m, 24H), 1.27-1.22 (m, 1H).

11B NMR (128 MHz, CDCl3) δ 0.74 (t, J=32.7 Hz).

13C NMR (151 MHz, CDCl3) δ 170.0, 164.0, 163.4, 156.9, 151.5, 150.6, 150.0, 149.0, 142.6, 138.5, 138.1, 137.8, 136.0, 134.5, 131.2, 124.6, 124.3, 122.1, 81.6, 55.2, 54.6, 28.2, 15.3, 14.7.

19F NMR (376 MHz, CDCl3) δ-145.46 −−146.69 (m).

### Preparation of Compound 8

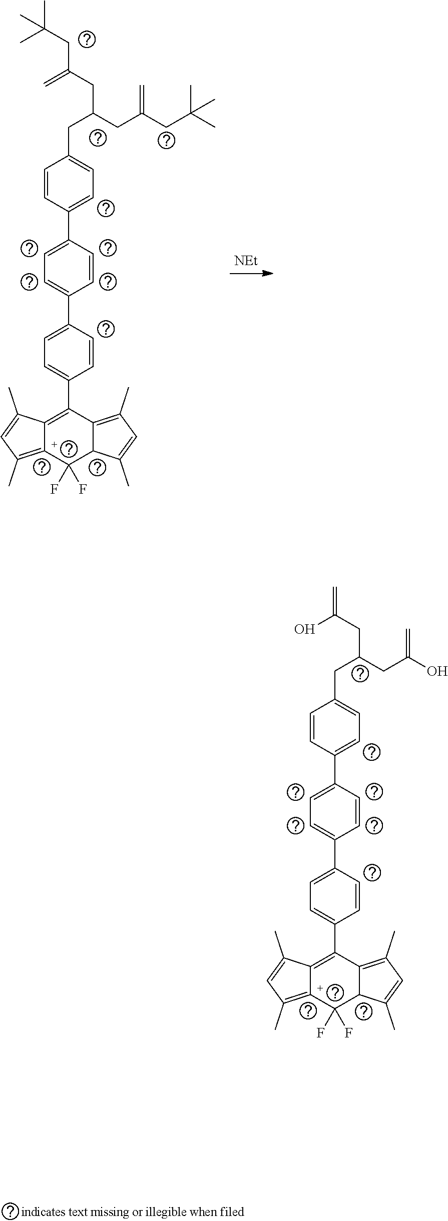

Compound 6 (31.6 mg, 42.7 μmol, 1.0 equiv.) was dissolved in 7 mL of 4 M HCl in dioxane and stirred for 3.5 hours. The solvent was removed under reduced pressure furnishing a red solid. The crude product was redissolved in 5 mL of MeCN:H2O (1:1 v/v, 0.1% TFA) and purified by preparative HPLC furnishing a red solid (16.7 mg, 26.6 μmol, 62%).

1H NMR (600 MHz, DMSO) b 12.43 (s, 2H), 9.02 (dd, J=2.2, 0.9 Hz, 1H), 8.91 (d, J=2.0 Hz, 1H), 8.81 (dd, J=8.0, 0.9 Hz, 1H), 8.63-8.59 (m, 1H), 8.30 (dd, J=8.0, 2.2 Hz, 1H), 8.15 (dd, J=8.1, 2.2 Hz, 1H), 6.27 (s, 2H), 4.06 (s, 2H), 3.52 (s, 4H), 1.45 (s, 6H). Two CH₃-groups were under the residual solvent peak.

13C NMR (151 MHz, DMSO) b 172.2, 163.2, 162.9, 155.8, 150.9, 150.7, 149.3, 148.9, 142.6, 138.2, 138.1, 137.7, 137.2, 132.9, 130.7, 124.2, 124.1, 122.0, 54.5, 54.0, 14.8, 14.3.

19F NMR (376 MHz, DMSO) δ-143.53 (q, J=31.6 Hz).

### Example 2—In Vivo Click Performance Using in-House Developed Blocking Assay

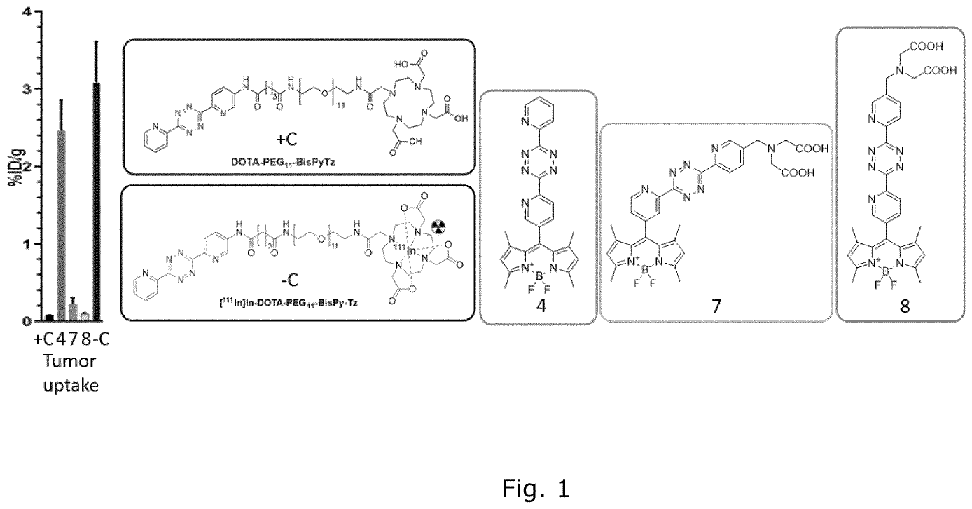

This assay employs anti-TAG72 mAb CC49 modified with TCO-tags (CC49-TCO) and [111In]In-DOTA-PEG11-BisPy-Tz (FIG. 1) with well-established performance in pretargeted imaging of TAG72 expressing LS174 tumor xenografts.

### Materials and Methods

LS174T xenograft-bearing mice are injected with CC49-TCO. After allowing for accumulation for 72 h, the tetrazine to be evaluated is injected via the tail vein, followed by [111In]In-DOTA-PEG11-BisPy-Tz after 1 h. in vivo performance of the tetrazine to be evaluated can therefore be correlated to tumor uptake of [111In]In-DOTA-PEG11-BisPy-Tz after 22 h. Increased in vivo performance is reflected by lower tumor uptake (see FIG. 1).

### Results and Discussion

FIG. 1 shows the in vivo click performance of the compounds 4, 7, and 8. Further, a negative control (−C) and a positive control (+C) are included. The positive control represents non-radioactive DOTA-PEG11-DOTA-BisPyTz injected in the same dose as compounds 4, 7 and 8 before the injection of radiolabelled [111In]-In-DOTA-PEG11-DOTA-BisPyTz to evaluate the blocking effect.

The negative control represents no injection of any blocking compound, but only radiolabelled [111In]-In-DOTA-PEG11-DOTA-BisPyTz, thus represents no blocking effect.

As shown in FIG. 1, the synthesized polar BODIPY-tetrazines compound 7 and 8 (of example 1) showed excellent blocking effects of 93 and 98%, respectively—following the trend observed in previous studies. The polarity allows for successful in vivo pretargeted fluorescence and PET imaging of LS174T tumor xenografts using CC49-TCO antibodies.

### Example 3—Pretargeted Fluorescence Imaging

Pretargeted imaging of compound 8 of Example 1 was tested in vivo.

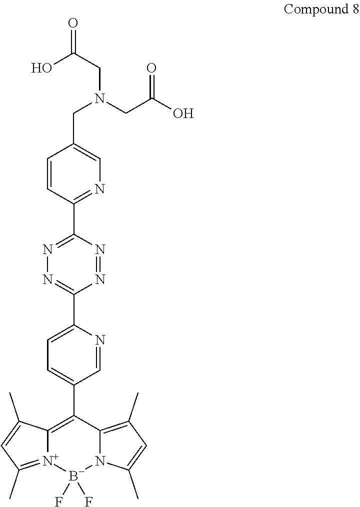 [SMILES: CC1=CC(C)=[N+]2C1=C(c1ccc(-c3nnc(-c4ccc(CN(CC(=O)O)CC(=O)O)cn4)nn3)nc1)c1c(C)cc(C)n1[B-]2(F)F]

2,2′-(((6-(6-(5-(5,5-difluoro-1,3,7,9-tetramethyl-5H-4λ4,5AN4-dipyrrolo[1,2-c:2′,1′-f][1,3,2]diazaborinin-10-yl)pyridin-2-yl)-1,2,4,5-tetrazin-3-yl)pyridin-3-yl)methyl)azanediyl)diacetic acid

### Materials and Methods

Pretargeted imaging of compound 8 of Example 1 was tested in vivo using the monoclonal antibody CC49 targeting TAG-72 in human colorectal cancer LS174T xenograft tumors. Tumors were established in 7-8 week old Balb/c nude mice and after 10 days, the animals were injected i.v. with either 50 μg (0.33 nmol) TCO-modified CC49 (CC49-TCO, ~3.5 TCO/mAb), or non-modified CC49 (control) (n=3 per group). After 72 hours, compound 8 (~100 equivalents per TCO, 86.5 μg, 116.7 nmol) in 100 μL of PBS (pH=7.4) was injected i.v.. One hour post injection, selected organs were harvested for ex vivo fluorescence measurements using a Amersham Typhoon 5 laser scanner platform (GE healthcare Europe) using a 488 nm excitation laser and 525/20 emission filter.

### Results and Discussion

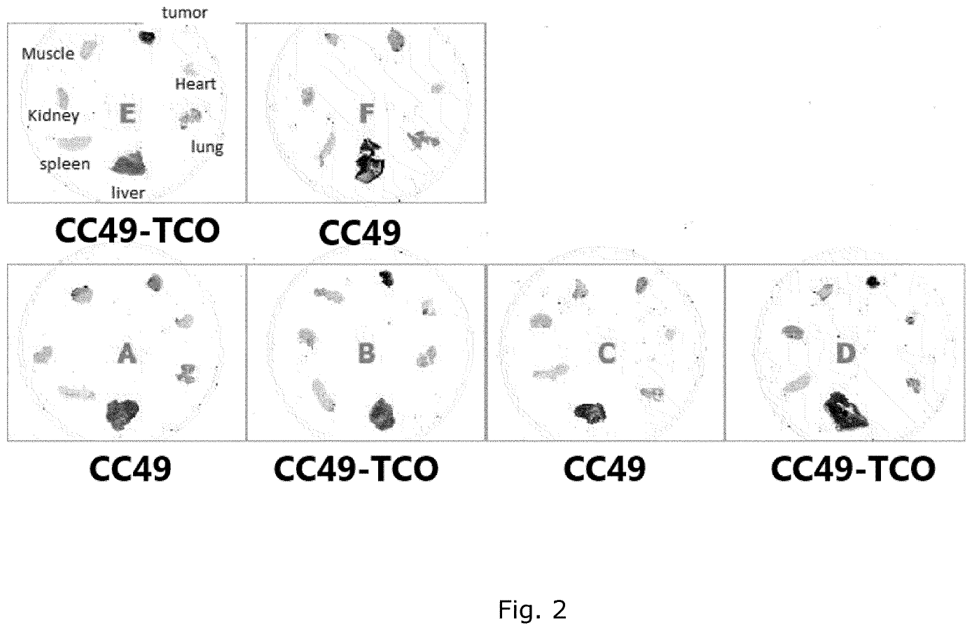

As seen in FIG. 2, the TCO-modified CC49 antibody causes a higher fluorescence in tumor. This is indicative of the successful click reaction in vivo, which can further increase the tumor to background ratios through the turn-on properties of the fluorophore.

### REFERENCES

(1) Carlson, J. C. T.; Meimetis, L. G.; Hilderbrand, S. A.; Weissleder, R. BODIPY-Tetrazine Derivatives as Superbright Bioorthogonal Turn-on Probes. Angew. Chemie—Int. Ed. 2013, 52 (27), 6917-6920. https://doi.org/10.1002/anie.201301100.
(2) Liu, S.; Lin, T. P.; Li, D.; Learner, L.; Shan, H.; Li, Z.; Gabbaï, F. P.; Conti, P. S. Lewis Acid-Assisted Isotopic 18F-19F Exchange in BODIPY Dyes: Facile Generation of Positron Emission Tomography/Fluorescence Dual Modality Agents for Tumor Imaging. Theranostics. 2013, pp 181-189. https://doi.org/10.7150/thno.5984.
(3) Stéen, E. J. L.; Jørgensen, J. T.; Denk, C.; Battisti, U. M.; Nørregaard, K.; Edem, P. E.; Bratteby, K.; Shalgunov, V.; Wilkovitsch, M.; Svatunek, D.; Poulie, C. B. M.; Hvass, L.; Sim6n, M.; Wanek, T.; Rossin, R.; Robillard, M.; Kristensen, J. L.; Mikula, H.; Kjaer, A.; Herth, M. M. Lipophilicity and Click Reactivity Determine the Performance of Bioorthogonal Tetrazine Tools in Pretargeted in Vivo Chemistry. ACS Pharmacol. Transl. Sci. 2021, 4 (2), 824-833. https://doi.org/10.1021/acsptsci.1c00007.

1. A compound represented by the formula (1): 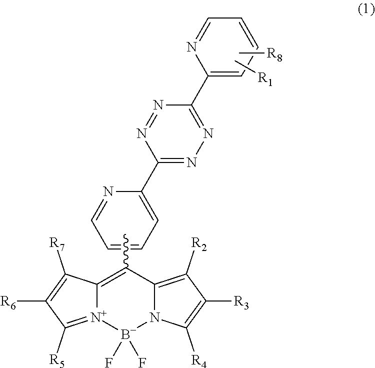
wherein R₁ is a hydrogen, —CH₂N(CH₂COOH)₂, a hydroxy group, a sulfonamide, a sulfonic acid or its salt, a carboxyl group, a sulfonyl group, amine, a substituted amine with 1-5 polyethylene glycol unit(s), a —(O—CH₂—CH₂)₁₋₅—OCH₂—COOH, or alkyl, preferably R₁ is in para position,
R₂ is a hydrogen or alkyl,
R₃ is a hydrogen, alkyl, a nitro group, a cyano group, a sulfonic acid or its salt, a carboxylic acid, an amide, a sulfonamide, —CH₂N(CH₂COOH)₂, a halogen or a radioactive halogen, such as fluorine-18, iodine-123, iodine-124, iodine-125, iodine-131, astatine-211,
R₄ is hydrogen, alkyl, —CH═CH(C₆H₄)R_{4′}, wherein R_{4′} is —N(R_{4″}R_{4′″})₂ or —OR_{4″}, wherein R_{4″} and R_{4′″} are independently alkyl, —CH₂COOH, —(CH₂—CH₂—O)₁₋₅—CH₂—COOH, 1-5 polyethylene units, fluorine-18 or a fluorine-19 labelled groups such as (CH₂)₁₋₆—F,
R₅ is hydrogen, alkyl, —CH═CH(C₆H₄)R_{5′}, wherein R_{5′} is —N(R_{5′} R_{5′″})₂ or —OR_{5″}, wherein R_{5″} and R_{5′″} are independently alkyl, —CH₂COOH, —(CH₂—CH₂—O)₁₋₅—CH₂—COOH, 1-5 polyethylene units, fluorine-18 labelled group, or fluorine-19 labelled group such as (CH₂)₁₋₆—F,
R₆ is a hydrogen, alkyl, a nitro group, a cyano group, a sulfonic acid, a sulfonic acid salt, a carboxylic acid, an amide, a halogen, a sulfonamide, or —CH₂N(CH₂COOH)₂,
R₇ is a hydrogen or alkyl, and
R₈ is selected hydrogen, a fluorine-18 or fluorine-19 labelled groups such as —O—(CH₂)₁₋₆—F and —(CH₂)₁₋₆—F, a halogen or a radioactive halogen, such as fluorine-18, iodine-123, iodine-124, iodine-125, iodine-131, or astatine-211;
wherein the wavy bond between the pyrridine and the [1,3,2]diazaborinine is in meta or para position on the pyrridine.

2. The compound according to claim 1, wherein the compound is represented by the formula (2): 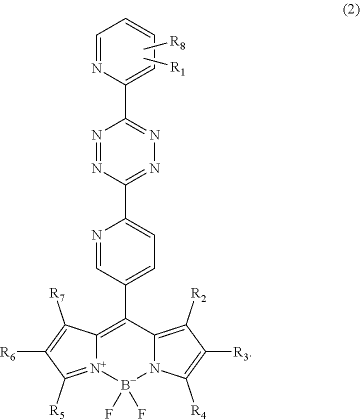
wherein.

3. The compound according to claim 1, wherein the compound is represented by the formula (3):

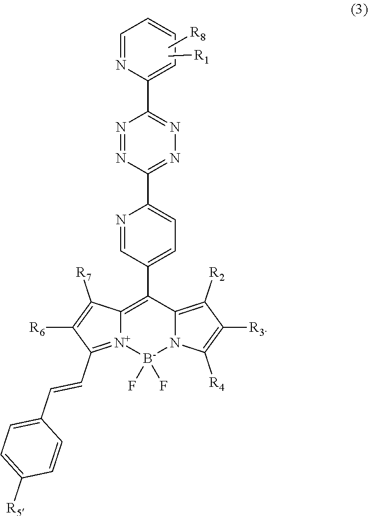

4. The compound according to claim 1, wherein the compound is represented by the formula (4):

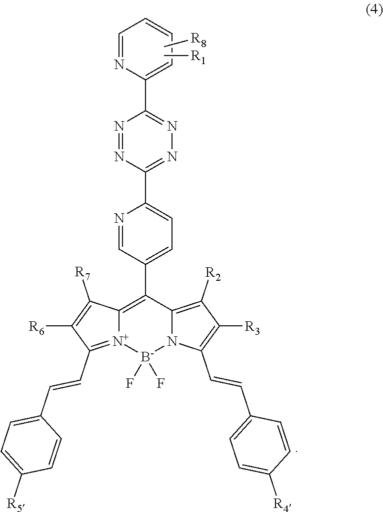

5. The compound according to a claim 1, wherein the compound is represented by the formula (5):

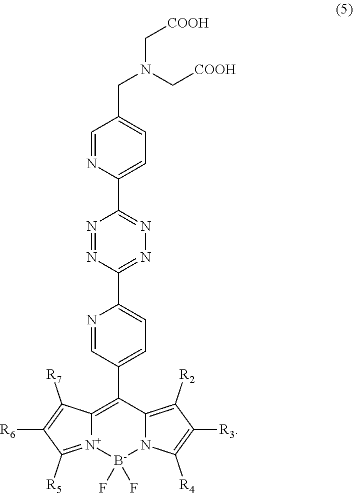

6. The compound according to claim 1, wherein the compound is represented by the formula (6):

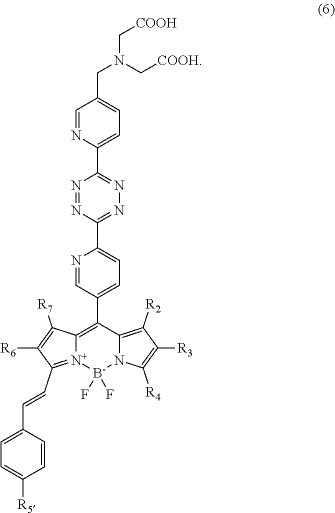

7. The compound according to a claim 1, wherein the compound is represented by the formula (7):

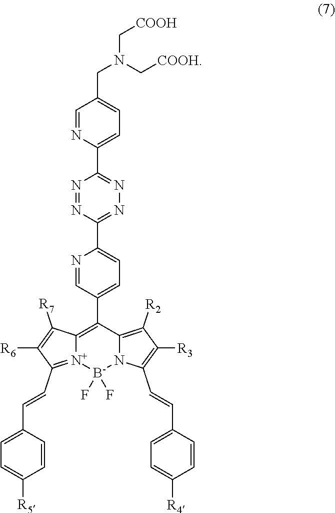

8. The compound according to claim 1, wherein the compound is represented by the formula (8):

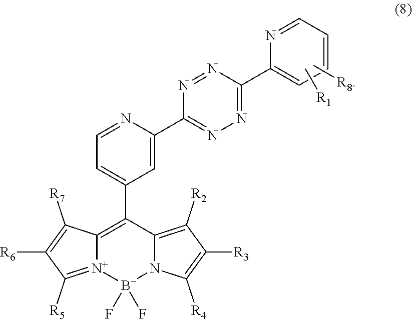

9. The compound according to claim 1, wherein the compound is represented by the formula (9):

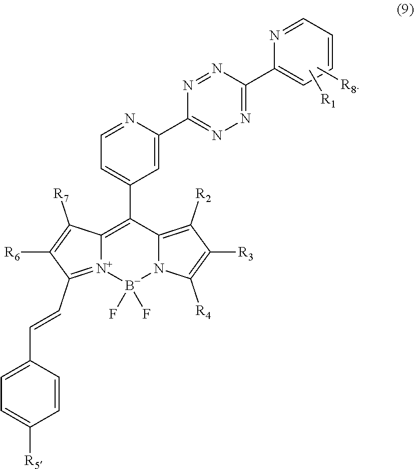

10. The compound according to a claim 1, wherein the compound is represented by the formula (10):

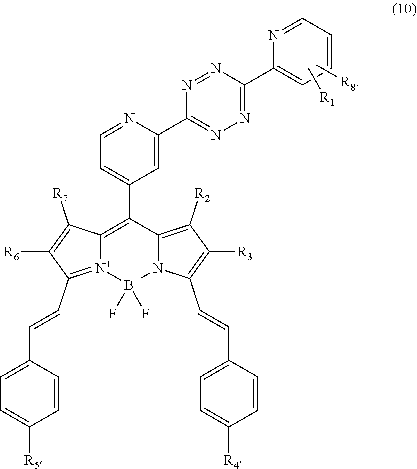

11. The compound according to a claim 1, wherein the compound is represented by the formula (11).

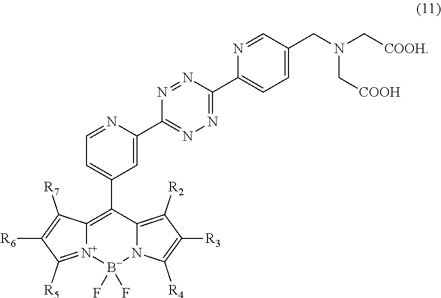

12. The compound according to claim 1, wherein the compound is represented by the formula (12):

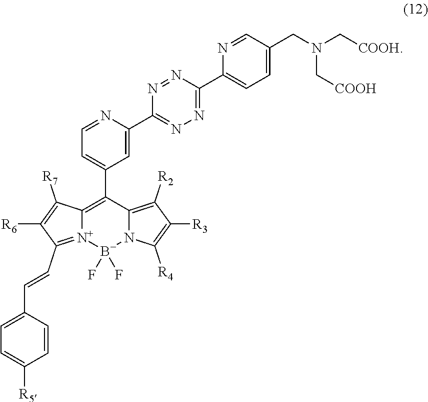

13. The compound according to a claim 1, wherein the compound is represented by the formula (13):

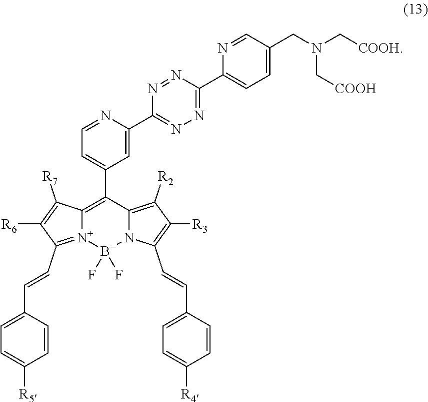

14. The compound according to claim 1, wherein the compound is (a), (b), (c), (d), (e), (f), (g), (h), (i), (j), (k), or (l):

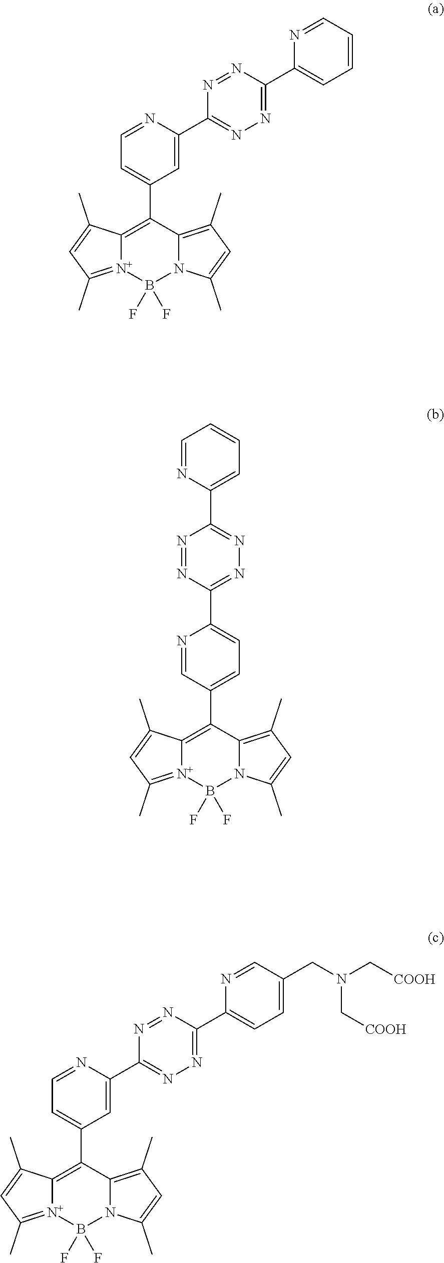

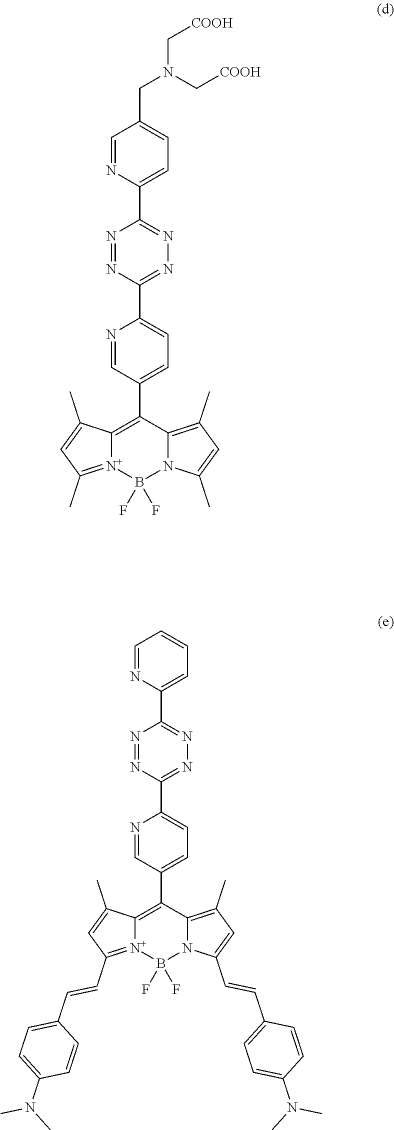

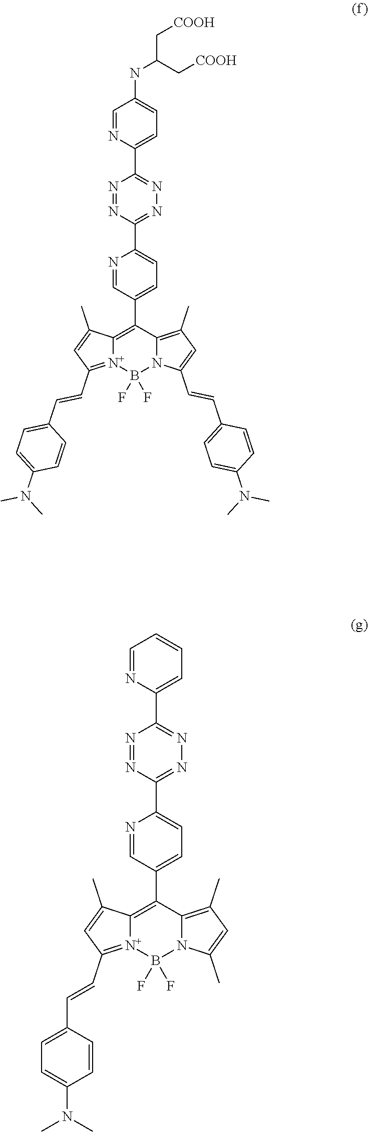

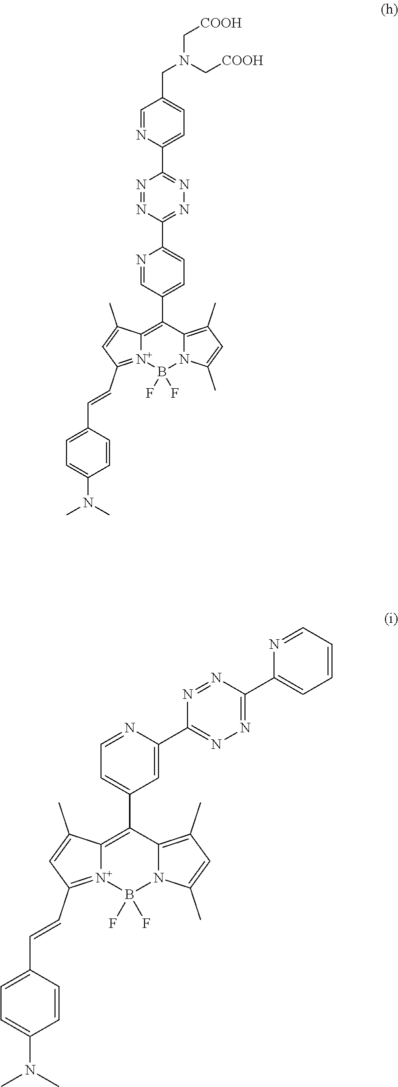

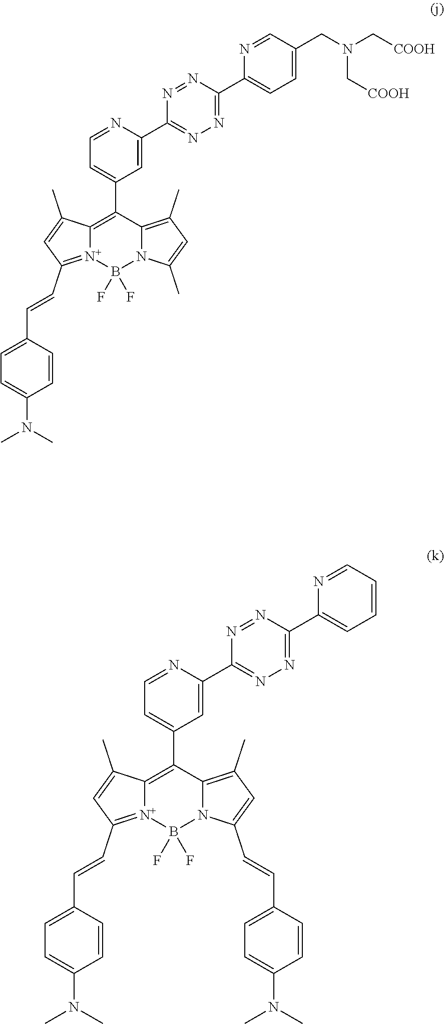

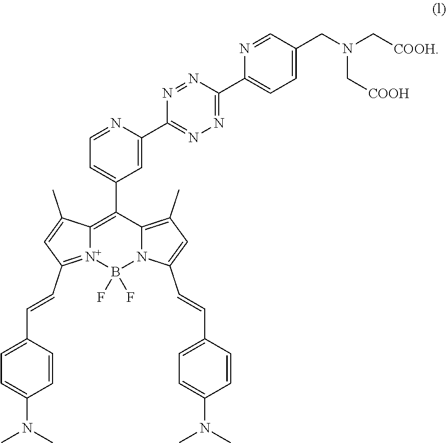

15. The compound according to claim 1 for use in PET imaging, SPECT imaging and/or diagnostics.

16. The compound according to claim 1 for use in intraoperative guided surgery.

17. The compound according to claim 1 for use in photodynamic or photothermal therapy.

18. A method of imaging a biological structure of a subject, the method comprising
administering a vector and the compound according to claim 1; and
imaging a contact between the vector and the compound in the biological structure,
the subject optionally being a mammal, and
the imaging optionally being PET or SPECT imaging.

19. The method according to claim 18, wherein the vector is a labelled vector and, wherein the labelled vector is a trans-cyclooctene (TCO)-labelled antibody, nanobody, affibody, single-chain variable fragment, peptide or small molecule, such as CC49-TCO, trastuzumab-TCO, PSMA-TCO or AE105-TCO (TCO-D-Cha-F-s-r-Y-L-W-S).

20. The method according to claim 18, wherein the biological structure is a tumor.

21-22. (canceled)
